(19) United States
(12) Patent Application Publication
(10) Pub. No.: US 2014/0135683 A1
(43) Pub. Date: May 15, 2014

(54) SYSTEMS, COMPONENTS, AND METHODS FOR EXPRESSING BREAST MILK

(71) Applicant: INFANTINO, LLC, Streetsboro, OH (US)

(72) Inventors: John L. Hradisky, Aurora, OH (US); Maureen O'Connor Salazar, Chicago, IL (US); Colette Gartner Cosky, San Diego, CA (US)

(73) Assignee: INFANTINO, LLC, Streetsboro, OH (US)

(21) Appl. No.: 13/675,402

(22) Filed: Nov. 13, 2012

(51) Int. Cl. A61M 1/06; F16L 15/00

(52) U.S. Cl. CPC A61M 1/06; F16L 15/00
USPC 604/74; 285/390

(57) ABSTRACT

Systems, components and methods for expressing breast milk are described herein. An adapter includes a first connector portion, a second connector portion and a transition portion extending between the first connector portion and the second connector portion. At least the first connector portion and the transition portion cooperate to define an interior chamber configured to receive fluid. The first connector portion is configured for connection to a pump assembly, and the second connector portion is configured for connection, via a plurality of internal threads, to a spout of a fluid container.

### TECHNICAL FIELD

The present application relates to systems, components, and methods for expressing breast milk.

### BACKGROUND

Mothers often choose to breast-feed their infants for a variety of reasons, e.g., to enhance the infant's immune system, to provide nutrients to the infant without the additives present in baby formula, and strengthening the bond between the mother and infant. In some instances, it can be desirable to express the breast milk, i.e., to obtain milk from the breast without the infant suckling, e.g., to refrigerate the breast milk for later use so that the infant can still have the benefits of breast milk when someone other than the mother is caring for the infant. Systems for expressing breast milk are known.

### SUMMARY

According to one embodiment, an adapter includes a first connector portion, a second connector portion and a transition portion extending between the first connector portion and the second connector portion. The first connector portion includes a first inner surface and a first outer surface. The second connector portion includes a second inner surface, a second outer surface, and a plurality of internal threads. The transition portion includes a third inner surface and a third outer surface. The third inner surface of the transition portion tapers inwardly toward the second inner surface of the second connector portion. At least the first connector portion and the transition portion cooperate to define an interior chamber configured to receive fluid. The first connector portion is configured for connection to a pump assembly to facilitate establishing at least intermittent fluid communication between an outlet passage of a pump assembly and the interior chamber. The second connector portion is configured for connection, via the plurality of internal threads, to a spout of a fluid container to facilitate establishing fluid communication between the interior chamber and a chamber defined by a fluid container.

According to another embodiment, a fluid container includes a bag that includes a first longitudinal centerline. The bag is at least partially collapsible, and is expandable. The fluid container also includes a spout attached to the bag and offset from the first longitudinal centerline. The spout includes a plurality of external threads and a second longitudinal centerline spaced from the first longitudinal centerline and parallel with the first longitudinal centerline. The spout defines a fluid passage. The bag also includes at least one plastic sheet attached to the spout, and defines an interior chamber in fluid communication with the fluid passage. The interior chamber is otherwise sealed.

According to another embodiment, a subassembly of a system for expressing breast milk includes an adapter that includes a first connector portion, a second connector portion, and a transition portion extending between the first connector portion and the second connector portion. The first connector portion includes a first inner surface and a first outer surface. The second connector portion includes a second inner surface, a second outer surface, and a plurality of internal threads. The transition portion includes a third inner surface and a third outer surface. The third inner surface of the transition portion tapers inwardly toward the second inner surface of the second connector portion. At least the first connector portion and the transition portion cooperate to define an interior chamber. The subassembly also includes a fluid container that is threadedly connected to the adapter. The fluid container includes a bag and a spout attached to the bag. The bag is at least partially collapsible, and is expandable. The bag defines a fluid chamber and the spout defines a flow passage in fluid communication with the fluid chamber. The spout includes a plurality of external threads. The first connector portion of the adapter is configured for connection to a pump assembly to facilitate establishing at least intermittent fluid communication between an outlet passage of a pump assembly and the interior chamber of the adapter. The plurality of internal threads of the second connector portion of the adapter is engaged with the plurality of external threads of the spout of the fluid container such that fluid communication is established between the interior chamber defined by the adapter and the fluid chamber defined by the bag of the fluid container, via the flow passage defined by the spout.

According to another embodiment, a subassembly of a system for expressing breast milk includes a hood that defines an extraction chamber, and a housing integral with the hood. The housing defines at least one flow passage. The subassembly also includes an outlet structure integral with the housing and defining an outlet passage, and a collar that is integral with the housing. The at least one flow passage defined by the housing establishes fluid communication between the outlet passage and the extraction chamber. The subassembly also includes an adapter that includes a first connector portion, a second connector portion, and a transition portion extending between the first connector portion and the second connector portion. The first connector portion includes a first inner surface and a first outer surface. The second connector portion includes a second inner surface, a second outer surface, and a plurality of internal threads. The transition portion includes a third inner surface and a third outer surface. The third inner surface of the transition portion tapers inwardly toward the second inner surface of the second connector portion. At least the first connector portion and the transition portion cooperate to define an interior chamber. The first connector portion of the adapter is connected to the collar to facilitate establishing at least intermittent fluid communication between the outlet passage defined by the outlet structure and the interior chamber defined by the adapter, during operation of a breast pump. The plurality of internal threads of the second connector portion is configured for engagement with a plurality of external threads of a spout of a fluid container.

According to another embodiment, a system for expressing breast milk includes a breast pump assembly that includes a hood defining an extraction chamber, a first housing integral with the hood, and an outlet structure integral with the first housing and defining an outlet passage in fluid communication with the extraction chamber. The breast pump assembly also includes a breast pump in fluid communication with each of the extraction chamber and the outlet passage. The system also includes an adapter that includes a first connector portion, a second connector portion, and a transition portion extending between the first connector portion and the second connector portion. At least the first connector portion and the transition portion cooperate to define an interior chamber. The system further includes a fluid container that includes a bag and a spout attached to the bag. The bag is at least partially collapsible, and is expandable. The bag defines a fluid chamber, and the spout defines a flow passage in fluid communication with the fluid chamber. The first connector portion of the adapter is connected to the breast pump assembly, and the second connector portion of the adapter is threadedly connected to the spout of the fluid container, such that the fluid chamber defined by the fluid container is in at least intermittent fluid communication with the outlet passage defined by the outlet structure of the breast pump assembly, during operation of the breast pump.

According to another embodiment, a method of expressing breast milk includes pumping breast milk into and through a flow passage defined by a threaded spout of a fluid container, and into a fluid chamber defined by a bag of the fluid container. The threaded spout is attached to the bag. The method also includes closing the fluid chamber by threading a cap onto the threaded spout of the fluid container, and storing the fluid container in a refrigerated space, for later use.

### BRIEF DESCRIPTION OF THE DRAWINGS

Various embodiments will become better understood with regard to the following description, appended claims and accompanying drawings wherein:

FIG. 1 is a schematic representation of a system for expressing breast milk;

FIG. 2 is an exploded perspective view depicting selected components of the system of FIG. 1;

FIG. 3 is a perspective view depicting an adapter, according to one embodiment, of the system of FIG. 1;

FIG. 4 is a bottom plan view of the adapter of FIG. 3;

FIG. 5 is a top plan view of the adapter of FIG. 3;

FIG. 6 is a cross-sectional view taken along line 6-6 in FIG. 5;

FIG. 7 is a side elevational view depicting an adapter according to another embodiment;

FIG. 8 is a side perspective view depicting an adapter according to yet another embodiment;

FIG. 9 is a side perspective view depicting an adapter according to still another embodiment;

FIG. 10 is a side perspective view depicting an adapter according to yet another embodiment;

FIG. 11 is a side perspective view depicting an adapter according to still another embodiment;

FIG. 12 is a cross-sectional view depicting an adapter according to still another embodiment;

FIG. 13 is a side elevational view, partially cut away, depicting an adapter according to another embodiment;

FIG. 14 is an exploded, elevational view depicting an adapter according to another embodiment, in association with a portion of a breast pump assembly;

FIG. 15 is a front elevational view depicting the fluid container of the system of FIG. 1, and further depicting a cap and a nipple in association with, and spaced from, the fluid container;

FIG. 16 is a perspective view depicting a fluid container according to another embodiment, with a nipple secured to a spout of the fluid container;

FIG. 17 is an exploded, side elevational view depicting a fluid container according to another embodiment, in association with an adapter according to another embodiment, and a nipple according to another embodiment;

FIG. 18 is a cross-sectional view depicting the adapter of FIG. 17;

FIG. 19 is a side elevational view depicting the components of FIG. 17 in an assembled configuration, and depicting a receptacle surrounding a portion of the fluid container;

FIG. 20 is a bottom plan view depicting the assembly of FIG. 19;

FIG. 21 is an exploded perspective view depicting the fluid container of the system of FIG. 1, in association with a portion of a breast pump assembly according to another embodiment;

FIG. 22 is a perspective view depicting a person's hand placing the fluid container of the system of FIG. 1, with the fluid container partially filled with breast milk, and capped, into a refrigerator;

FIG. 23 is a perspective view depicting a person's hand holding a receptacle containing the fluid container of FIG. 22, under a stream of water to raise the temperature of the breast milk within the fluid container;

FIG. 24 is a right, front perspective view of a receptacle according to one embodiment;

FIG. 25 is a left, rear perspective view of the receptacle of FIG. 24;

FIG. 26 is a top plan view of the receptacle of FIG. 24;

FIG. 27 is a right, front perspective view of the receptacle of FIG. 24 in association with a fluid container, a nipple and an adapter, with the nipple shown cross-section; and

FIG. 28 is a fragmentary top plan view illustrating a portion of the assembly of FIG. 27, with the nipple and the adapter removed for clarity of illustration.

### DETAILED DESCRIPTION

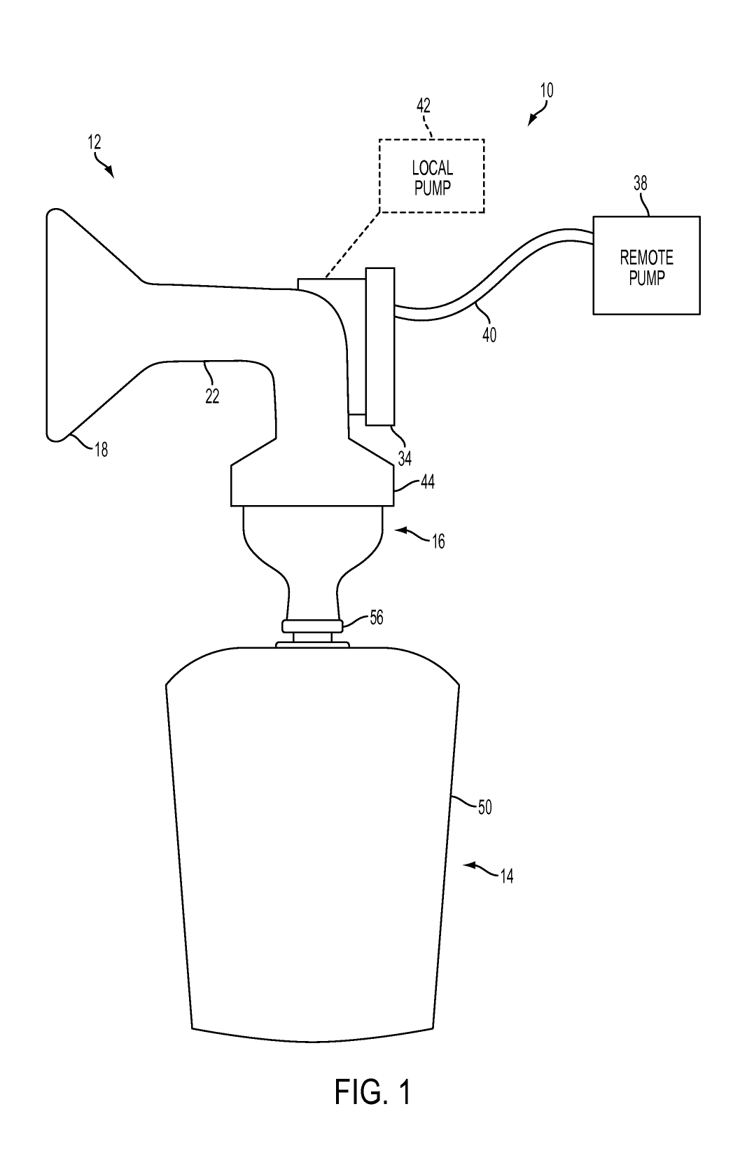

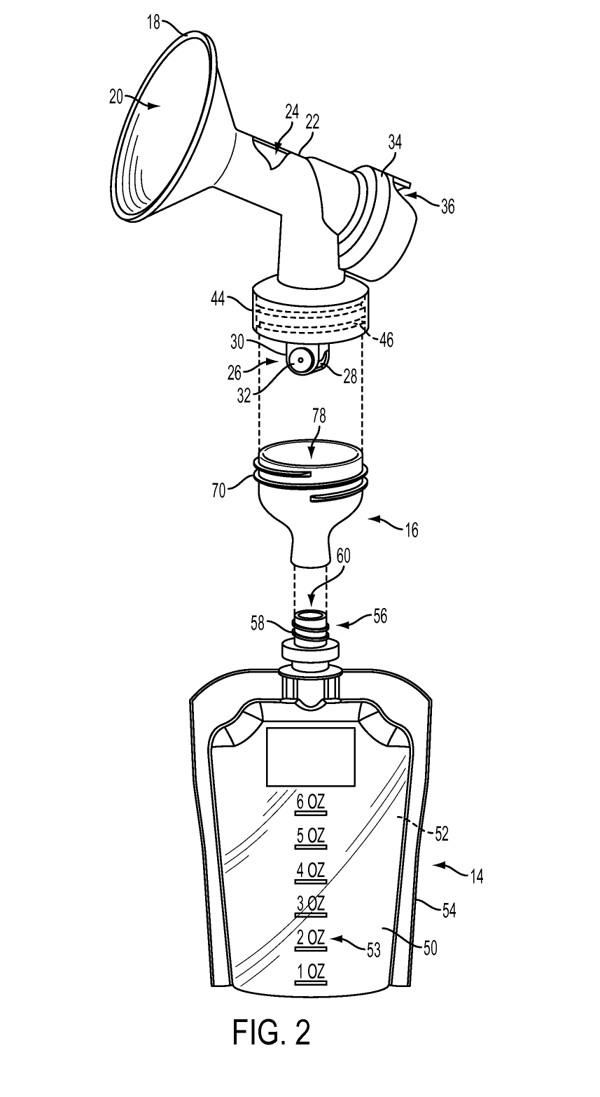

Referring to the drawings, wherein like numbers indicate the same or corresponding elements throughout the views, FIG. 1 illustrates a system 10 for expressing breast milk, according to one embodiment. System 10 can include a breast pump assembly 12, a fluid container 14, and an adapter 16, according to one embodiment. The adapter 16 can be connected to each of the breast pump assembly 12 and the fluid container 14. The breast pump assembly 12 can include a hood 18, which can define an extraction chamber 20 (FIG. 2). The hood 18 and extraction chamber 20 can be configured such that the hood 18 can engage a breast in at least substantially sealing engagement, with a portion of the breast, including the nipple, being disposed within the extraction chamber 20. As shown in FIG. 2, the breast pump assembly 12 can further include a first housing 22, which can define at least one flow passage 24, and an outlet structure 26, which can define an outlet passage 28. The first housing 22 can be integral with the hood 18, i.e., formed separately but in contact with the hood 18 and attached to the hood 18, or integrally formed with the hood 18 as a unitary structure. The outlet structure 26 can be attached to the first housing 22 in an interference fit, but can be selectively removed to facilitate cleaning of the extraction chamber 20. In one alternative embodiment, the outlet structure 26 can be integrally formed with the first housing 22 as a unitary component. The outlet structure 26 can include a housing 30 and a valve member 32. The valve member 32 can be a relatively thin and flexible diaphragm, which can be movable relative to the housing 30.

The breast pump assembly 12 can also include a second housing 34, which can be integral with the first housing 22, and can define a vacuum chamber 36 (FIG. 2). The breast pump assembly 12 can also include a breast pump. In one embodiment, the breast pump can be a remote pump 38, which can be remotely positioned relative to the remainder of the breast pump assembly 12, as schematically illustrated in FIG. 1. The remote pump 38 can be an electric pump and can be in fluid communication with the vacuum chamber 36, for example via a conduit 40 that can be connected at one end to the remote pump 38 and can be connected at an opposite end to the second housing 34. Alternatively, the breast pump of the breast pump assembly 12 can be a local pump 42, which can be locally mounted, for example, to the second housing 34, or another structure of the breast pump assembly 12. The local pump 42 can be in fluid communication with the vacuum chamber 36 defined by the second housing 34. In one embodiment, the local pump 42 can be a manual pump. In another embodiment, the local pump can be an electric pump. The breast pump assembly 12 can also include a collar 44, which can be integral with the first housing 22. In one embodiment, the collar 44 can include a plurality of internal threads 46 (FIG. 2).

The fluid container 14 can include a bag 50, which can define a fluid chamber 52. The bag 50 can be at least partially collapsible, and can be expandable, such that the fluid chamber 52 can have a predetermined, maximum volume. The fluid container 14 can also include a perimeter flange 54, which can extend at least partially around a perimeter of the bag 50. The fluid container 14 can also include a spout 56, which can be attached to the bag 50. The spout 56 can include a plurality of external threads 58, and can define a fluid passage 60 (FIG. 2). The fluid chamber 52 defined by the bag 50 can be in fluid communication with the fluid passage 60, and can be otherwise sealed, such that a caretaker using the fluid container 14 cannot create an opening (other than through the spout 56) without destroying the bag 50. The bag 50 can include at least one plastic sheet, using any suitable plastic material. In one embodiment, bag 50 can include two plastic sheets, which can be attached to one another and to the spout 56, e.g., using a heat-sealing process and/or adhesive. The two plastic sheets can cooperate to define the interior chamber 52 and can form at least a portion of the perimeter flange 54 of the fluid container 14. The bag 50 can also include graduated indicia, indicated generally at 53, which can indicate the volume of fluid, e.g., breast milk, within the fluid chamber 52, as measured in ounces or milliliters, for example.

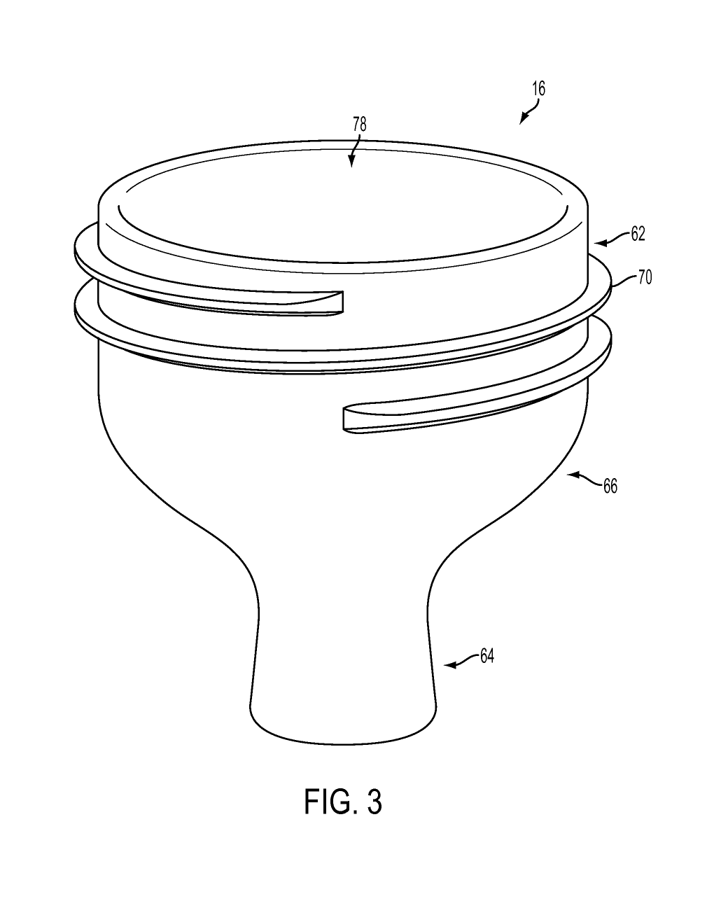

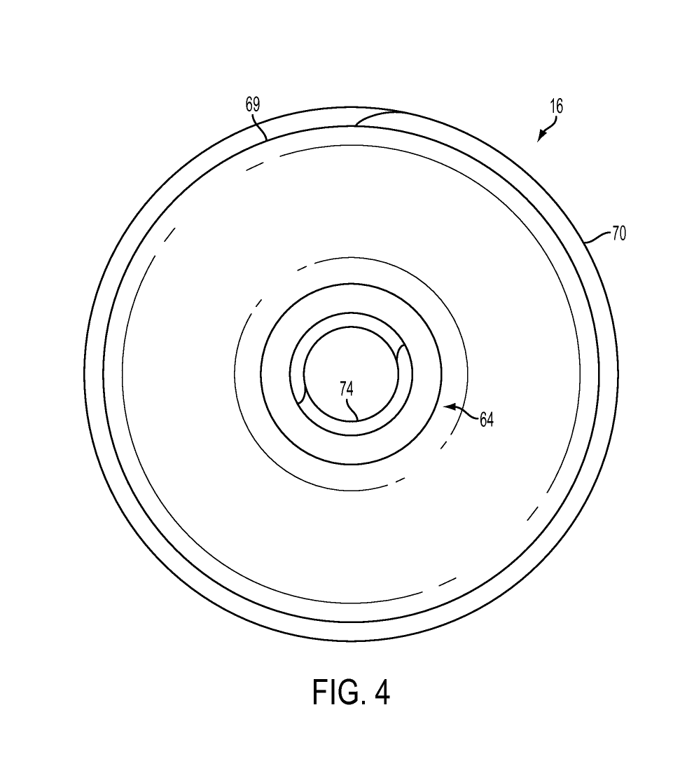

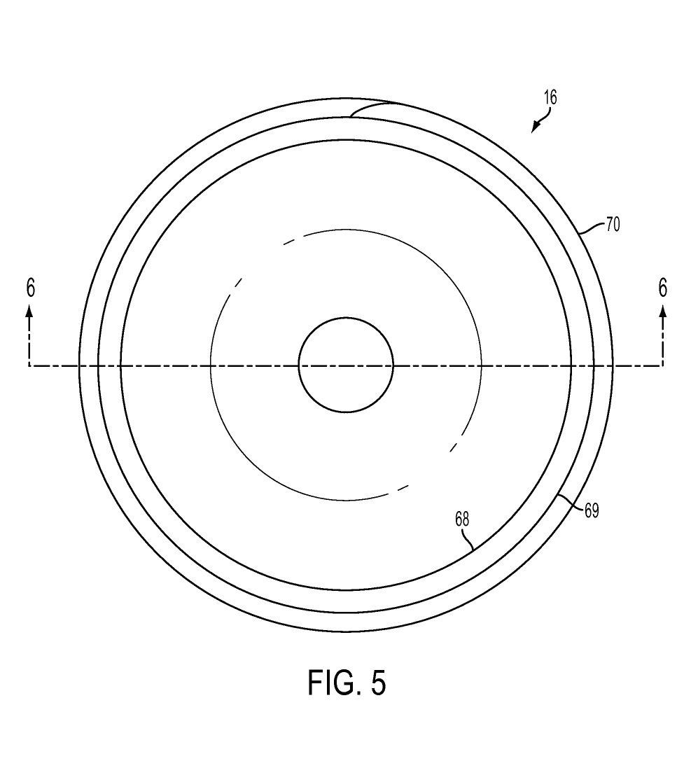

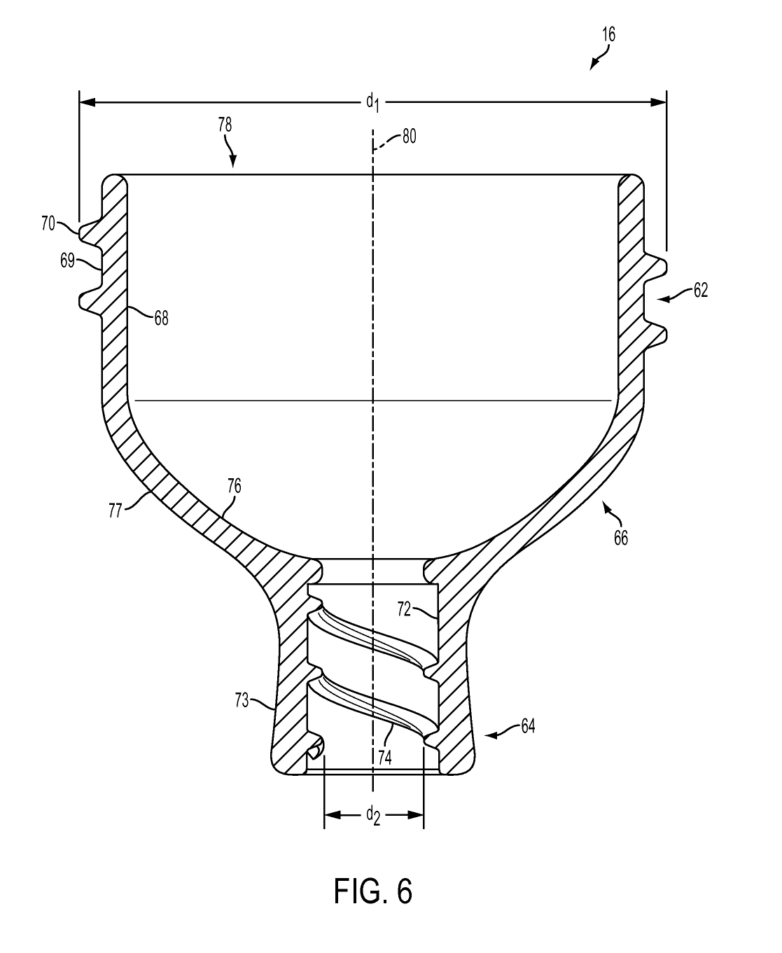

Referring to FIGS. 3-6, the adapter 16 can include a first connector portion 62, a second connector portion 64, and a transition portion 66, which can extend between the first connector portion 62 and the second connector portion 64. As shown in FIG. 6, the first connector portion 62 can include an inner surface 68, an outer surface 69, and a plurality of external threads 70, which can extend away from the outer surface 69. The second connector portion 64 can include an inner surface 72, an outer surface 73, and a plurality of internal threads 74. The external threads 70 can have a pitch diameter d₁ which can be greater than a pitch diameter d₂ of the internal threads 74, as shown in FIG. 6. It will be appreciated that a plurality of threads, as described herein, can include one or more raised or indented features that extend helically or generally helically about at least a significant portion of a circumference of an object, and in some cases more than once about a circumference of an object, in a continuous or discontinuous configuration.

The transition portion 66 of adapter 16 can include an inner surface 76 and an outer surface 77. As shown in FIG. 6, the inner surface 76 of the transition portion 66 can taper inwardly toward the inner surface 72 of the second connector portion 64. The inner surface 76 of the transition portion 66 can be generally cup-shaped and can blend smoothly with at least the inner surface 68 of the first connector portion 62 of adapter 16, as shown in FIG. 6. The outer surface 77 of the transition portion 66 can also be generally cup-shaped, as shown in FIG. 6. At least the first connector portion 62 and the transition portion 66 of adapter 16 can cooperate to define an interior chamber 78, which can be configured to receive fluid, for example, breast milk. The adapter 16 can include a longitudinal centerline 80. In one embodiment, the longitudinal centerline 80 can centrally bisect each of the first connector portion 62, the second connector portion 64 and the transition portion 66 of the adapter 16, as shown in FIG. 6.

The adapter 16 can be threadedly connected to each of the fluid container 14 and the breast pump assembly 12. In one embodiment, the internal threads 74 of the second connector portion 64 of adapter 16 can engage the external threads 58 of spout 56 of fluid container 14, such that the second connector portion 64 of the adapter 16 is connected to the fluid container 14, and the fluid passage 60 defined by the spout 56 of fluid container 14 is in fluid communication with the interior chamber 78 defined by the adapter 16, which establishes fluid communication between the interior chamber 78 and the fluid chamber 52 defined by the bag 50 of the fluid container 14. The external threads 70 of the first connector portion 62 of the adapter 16 can be engaged with the internal threads 46 of the collar 44 of the breast pump assembly 12, such that the first connector portion 62 is connected to the collar 44, which connects the adapter 16 to the breast pump assembly 12.

When the first connector portion 62 is connected to the collar 44, at least a portion of the outlet structure 26 of the breast pump assembly 12, including the valve member 32, can be positioned within the interior chamber 78 defined by the adapter 16. As a result, the interior chamber 78 and the fluid chamber 52 defined by the bag 50 of the fluid container 14, can be in intermittent fluid communication with the outlet passage 28 defined by the outlet structure 26 of the breast pump assembly 12 during operation of the breast pump (e.g., 38, 42) of the breast pump assembly 12. During operation of either the remote pump 38 or the local pump 42, a negative pressure, or vacuum, can intermittently exist within the vacuum chamber 36, the outlet passage 28, flow passage 24 and the extraction chamber 20, of the breast pump assembly 12. When a negative pressure, or vacuum, exists within the vacuum chamber 36, outlet passage 28, flow passage 24 and extraction chamber 20, the valve member 32 can be forced into a closed position against the housing 30 of the outlet structure 26, and breast milk can be drawn into the extraction chamber 20, and into the outlet passage 28 via the flow passage 24. When the negative pressure, or vacuum, is released, the expressed, or extracted, breast milk can flow out of the outlet passage 28 into the interior chamber 78 defined by the adapter 16, under the action of gravity and/or as a result of intermittent positive pressure within the outlet passage 28 forcing the valve member 32 open. The expressed breast milk can then flow through the fluid passage 60 defined by spout 56 of fluid container 14, and into the fluid chamber 52 defined by the bag 50 of fluid container 14.

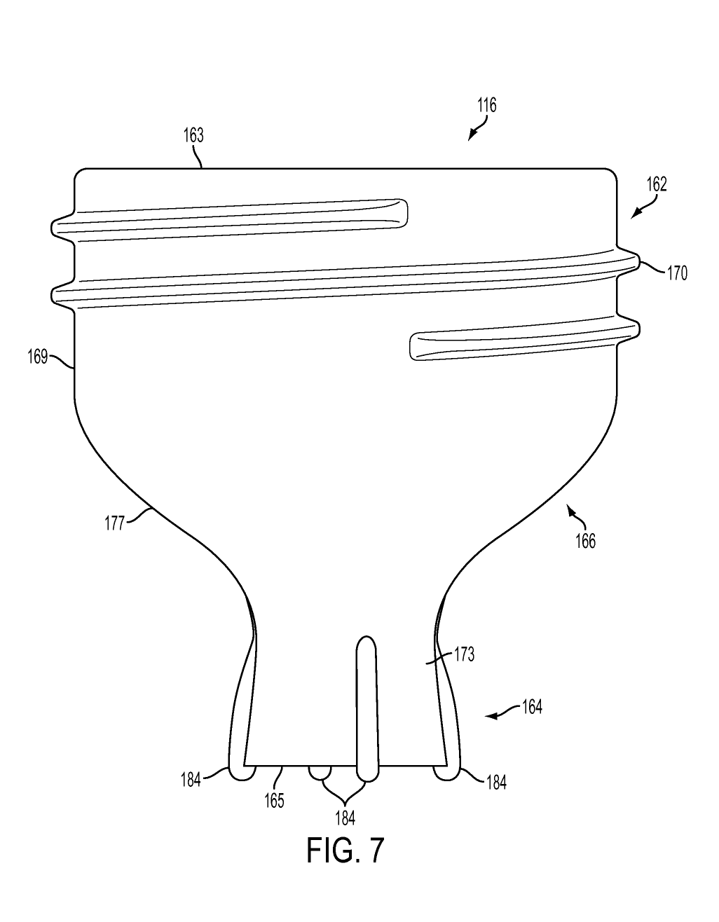

FIGS. 7-14 illustrate adapters according to other embodiments, which can be used in lieu of the adapter 16 in conjunction with the breast pump assembly 12 and fluid container 14, or in conjunction with breast pump assemblies and/or fluid containers according to other embodiments. FIG. 7 illustrates an adapter 116 according to another embodiment. Adapter 116 can be configured the same as, or similar to, adapter 16, except for the addition of a plurality of circumferentially spaced fins 184. Similar to the adapter 16, the adapter 116 can include a first connector portion 162, a second connector portion 164 and a transition portion 166, which can extend between the first connector portion 162 and the second connector portion 164. The first connector portion 162 can include an inner surface (not shown), an outer surface 169 and a plurality of external threads 170, which can extend outwardly from the outer surface 169. The first connector portion 162 can also include an annular proximal surface 163.

The second connector portion 164 can include an inner surface (not shown), an outer surface 173, and a plurality of internal threads (not shown). The second connector portion 164 can also include an annular distal surface 165. The transition portion 166 of adapter 116 can include an inner surface (not shown) and an outer surface 177. The inner surface of the transition portion 166 can taper inwardly toward the inner surface of the second connector portion 164, and can blend smoothly with at least the inner surface of the first connector portion 162. At least the first connector portion 162 and the transition portion 166 can cooperate to define an interior chamber (not shown) configured to receive fluid. Each of the fins 184 can be integral with at least the second connector portion 164, and can extend outwardly from the outer surface 173 of the second connector portion 164, as shown in FIG. 7. Each of the fins 184 can extend distally beyond the annular distal surface 165 of the second connector portion 164. As a result, the adapter 116 can be placed on a support surface (not shown), with the adapter 116 oriented as shown in FIG. 7, such that the fins 184 can contact the support surface and the annular distal surface 165 of the second connector portion 164 can be spaced from the support surface, which can facilitate preventing the second connector portion 164 from becoming contaminated.

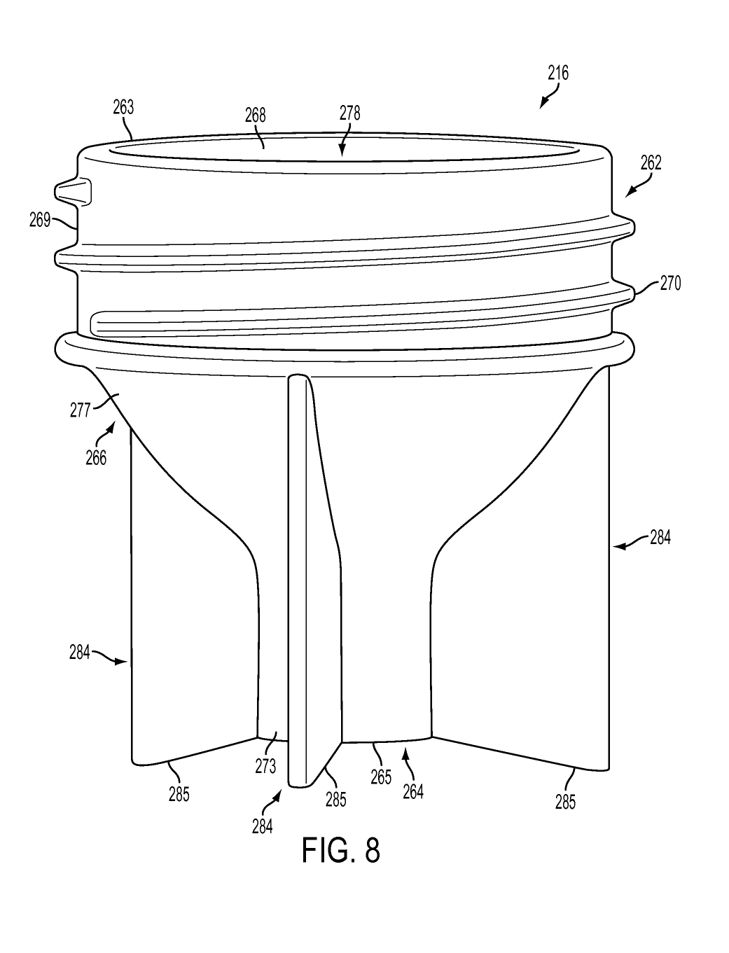

FIG. 8 illustrates an adapter 216 according to another embodiment. Adapter 216 can be configured the same as, or similar to, adapter 16, except for the addition of a plurality of circumferentially spaced fins 284. Similar to the adapter 16, the adapter 216 can include a first connector portion 262, a second connector portion 264 and a transition portion 266, which can extend between the first connector portion 262 and the second connector portion 264. The first connector portion 262 can include an inner surface 268, an outer surface 269, and a plurality of external threads 270, which can extend outwardly from the outer surface 269. The first connector portion 262 can also include an annular proximal surface 263.

The second connector portion 264 can include an inner surface (not shown), an outer surface 273, an annular distal surface 265, and a plurality of internal threads (not shown). The transition portion 266 of adapter 216 can include an inner surface (not shown) and an outer surface 277. The inner surface of the transition portion 266 can taper inwardly toward the inner surface of the second connector portion 264, and can blend smoothly with at least the inner surface of the first connector portion 262. At least the first connector portion 262 and the transition portion 266 can cooperate to define an interior chamber 278 configured to receive fluid. Each of the fins 284 can extend outwardly from each of the second connector portion 264 and the transition portion 266. In one embodiment, as shown in FIG. 8, each of the fins 284 can extend radially outwardly from each of the second connector portion 264 and the transition portion 266. Each of the fins 284 can include a distal surface 285, which can extend outwardly from the annular distal surface 265 of the second connector portion 264. In one embodiment, the distal surface 285 of each fin 284 can be linear, and can extend outwardly from the annular distal surface 265 of the second connector portion 264 such that the distal surface 285 of each of the fins 284 can be coplanar with the annular distal surface 265 of the second connector portion 264, as shown in FIG. 8. This configuration can enhance the stability of the adapter 216 when the adapter 216 is placed on a support surface, with the distal surface 285 of each of the fins 284, and the annular distal surface 265 of the second connector portion 264, contacting the support surface. Fins 284 can facilitate grasping the adapter 216.

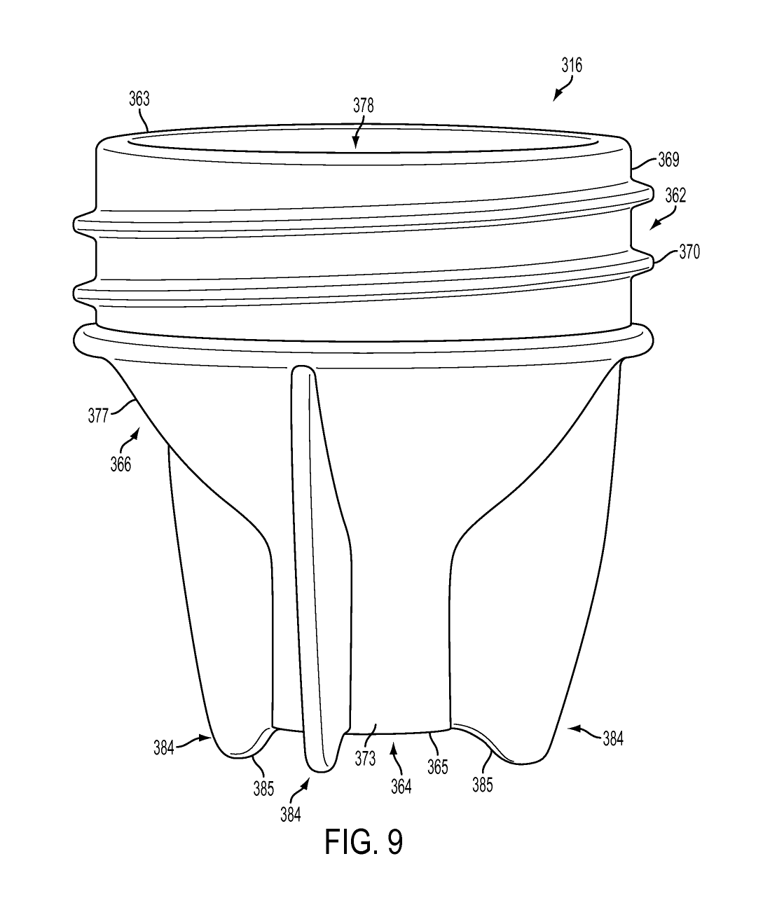

FIG. 9 illustrates an adapter 316, according to another embodiment. Adapter 316 can be configured the same as, or similar to, adapter 16, except for the addition of a plurality of circumferentially spaced fins 384. Similar to the adapter 16, the adapter 316 can include a first connector portion 362, a second connector portion 364 and a transition portion 366, which can extend between the first connector portion 362 and the second connector portion 364. The first connector portion 362 can include an inner surface (not shown), an outer surface 369 and a plurality of external threads 370, which can extend outwardly from the outer surface 369. The first connector portion 362 can also include an annular proximal surface 363.

The second connector portion 364 can include an inner surface (not shown), an outer surface 373, and a plurality of internal threads (not shown). The transition portion 366 can include an inner surface (not shown) and an outer surface 377. The inner surface of the transition portion 366 can taper inwardly toward the inner surface of the second connector portion 364, and can blend smoothly with at least the inner surface of the first connector portion 362. At least the first connector portion 362 and the transition portion 366 can cooperate to define an interior chamber 378, which can be configured to receive fluid. The second connector portion 364 can also include an annular distal surface 365. Each of the fins 384 can be integral with, and can extend outwardly from, each of the second connector portion 364 and the transition portion 366 of the adapter 316. In one embodiment, each of the fins 384 can extend radially outwardly from each of the second connector portion 364 and the transition portion 366. Each of the fins 384 can include a distal surface 385, which can extend outwardly from the second connector portion 364, as shown in FIG. 9. In one embodiment, the distal surface 385 can be arcuate, and can extend distally beyond the annular distal surface 365 of the second connector portion 364, as shown in FIG. 9. As a result, when the adapter 316 is placed on a support surface, the fins 384 can contact the support surface and the annular distal surface 365 of the second connector portion 364 can be spaced from the support surface, which can facilitate preventing the second connector portion 364 from becoming contaminated.

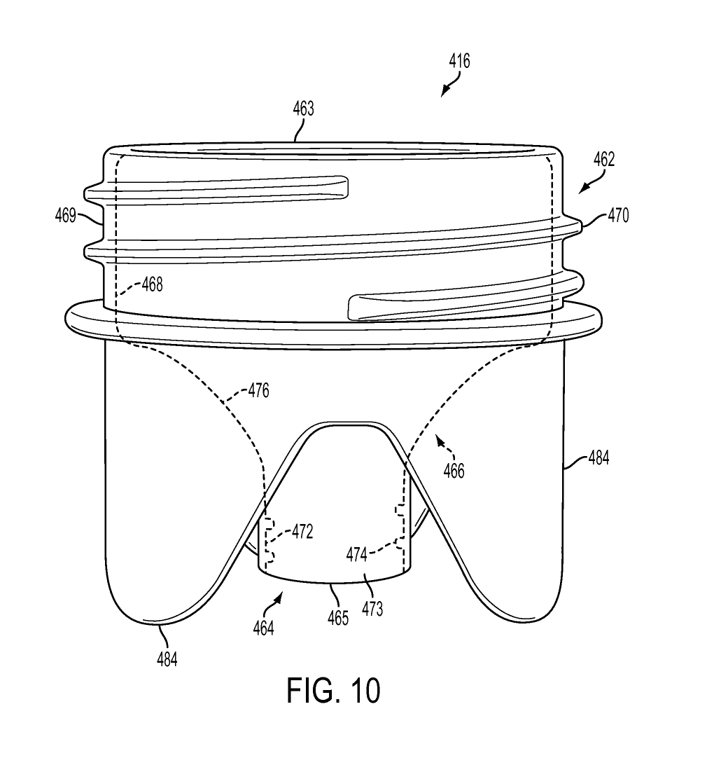

FIG. 10 illustrates an adapter 416, according to another embodiment. Adapter 416 can be configured the same as, or similar to, adapter 16, except for the addition of a plurality of circumferentially spaced fins 484. Similar to the adapter 16, the adapter 416 can include a first connector portion 462, a second connector portion 464, and a transition portion 466, which can extend between the first connector portion 462 and the second connector portion 464. The first connector portion 462 can include an inner surface 468, an outer surface 469 and plurality of external threads 470, which can extend outwardly from the outer surface 469. The first connector portion 462 can also include an annular proximal surface 463.

The second connector portion 464 can include an inner surface 472, an outer surface 473, an annular distal surface 465, and a plurality of internal threads 474. The transition portion 466 can include an inner surface 476 and an outer surface (not shown). The inner surface 476 of the transition portion 466 can taper inwardly toward the inner surface 472 of the second connector portion 464, and can blend smoothly with at least the inner surface 468 of the first connector portion 462. At least the first connector portion 462 and the transition portion 466 can cooperate to define an interior chamber that can be configured to receive fluid. Each of the fins 484 can be integral with, and can extend distally from, the first connector portion 462, and can extend distally beyond the annular distal surface 465 of the second connector portion 464, as shown in FIG. 10. As a result, when the adapter 416 is placed on a support surface, the fins 484 can contact the support surface and the annular distal surface 465 of the second connector portion 464 can be spaced from the support surface, which can facilitate preventing the second connector portion 464 from becoming contaminated. Adapters can be provided that include circumferentially spaced fins that are configured and positioned other than as shown in the embodiments of FIGS. 7-10.

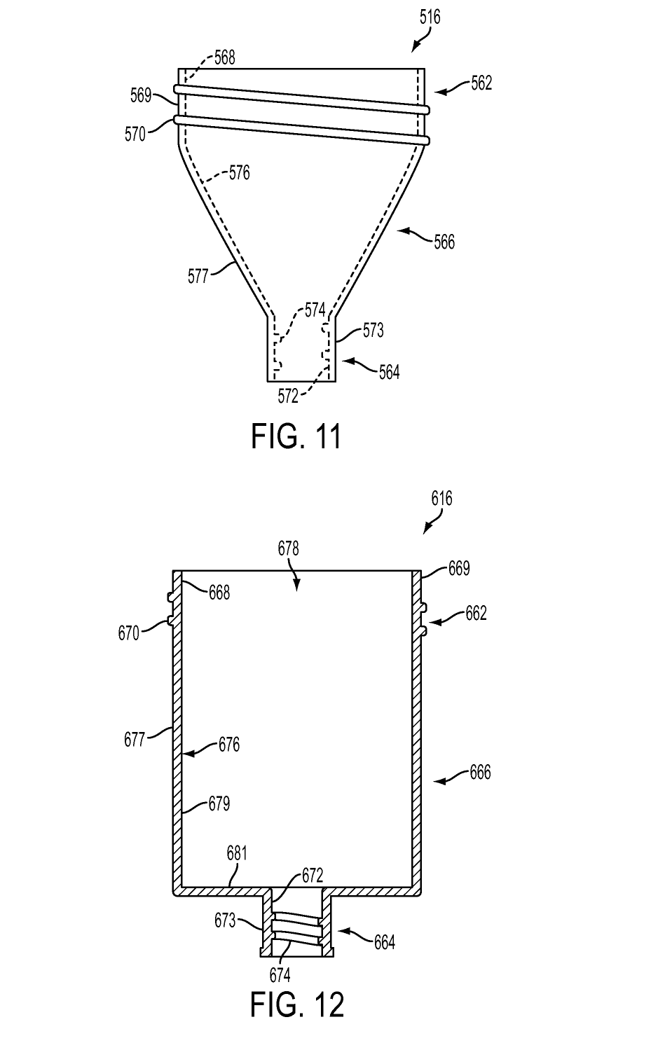

FIG. 11 illustrates an adapter 516 according to another embodiment. Similar to the adapter 16, the adapter 516 can include a first connector portion 562, a second connector portion 564, and a transition portion 566, which can extend between the first connector portion 562 and the second connector portion 564. The first connector portion 562 can include an inner surface 568, an outer surface 569, and a plurality of external threads 570, which can extend away from the outer surface 569. The second connector portion 564 can include an inner surface 572, an outer surface 573, and a plurality of internal threads 574. The external threads 570 can have a pitch diameter that can be greater than a pitch diameter of the internal threads 574, as shown in FIG. 11. The transition portion 566 can include an inner surface 576 and an outer surface 577. The inner surface 576 of the transition portion 566 can taper inwardly toward the inner surface 572 of the second connector portion 564. Unlike the adapter 16, each of the inner surface 576 and the outer surface 577 of the transition portion 566 can have a frusto-conical shape. At least the first connector portion 562 and the transition portion 566 can cooperate to define an interior chamber (not shown), which can be configured to receive fluid, for example, breast milk.

The adapter 516 can be threadedly connected to each of the fluid container 14 and the breast pump assembly 12. In one embodiment, the internal threads 574 of the second connector portion 564 can engage the external threads 58 of the spout 56 of the fluid container 14, and the external threads 570 of the first connector portion 562 can engage the internal threads 46 of the collar 44 of the breast pump assembly 12, such that the interior chamber (not shown) defined by the adapter 516 can be in fluid communication with the fluid chamber 52 defined by the bag 50 of the fluid container 14, and can be in at least intermittent fluid communication with the outlet passage 28 of the outlet structure 26 of the breast pump assembly 12.

FIG. 12 illustrates an adapter 616 according to another embodiment. Similar to adapter 16, the adapter 616 can include a first connector portion 662, a second connector portion 664, and a transition portion 666, which can extend between the first connector portion 662 and the second connector portion 664. The first connector portion 662 can include an inner surface 668, an outer surface 669, and a plurality of external threads 670. The second connector portion 664 can include an inner surface 672, an outer surface 673, and a plurality of internal threads 674. The transition portion 666 can include an inner surface 676 and an outer surface 677. Unlike the adapter 16, the inner surface 676 of the of the transition portion 666 does not taper inwardly toward the inner surface 672 of the second connector portion 664. Instead, the inner surface 676 can include a first portion 679, which can have a cylindrical shape, and can include a second portion 681, which can have an annular shape and can extend between the first portion 679 of the inner surface 676 of the transition portion 666, and the inner surface 672 of the second connector portion 674. In one embodiment, the first portion 679 of the inner surface 676 can be perpendicular to the second portion 681 of the inner surface 676, as shown in FIG. 12. At least the first connector portion 662 and the transition portion 666 can cooperate to define an interior chamber 678, which can be configured to receive fluid, for example, breast milk.

The plurality of internal threads 674 of the second connector portion 664 can be engaged with the external threads 58 of the spout 56 of fluid container 14, to connect the adapter 16 to the fluid container 14, such that the interior chamber 678 defined by the adapter 616 is in fluid communication with the fluid chamber 52 defined by the bag 50 of the fluid container 14. The plurality of external threads 670 of the first connector portion 662 can be engaged with the plurality of internal threads 46 of the collar 44 of the breast pump assembly 12, such that the interior chamber 678 defined by the adapter 616 can be in at least intermittent fluid communication with the outlet passage 28 defined by the outlet structure 26 of the breast pump assembly 12. As a result of the connection of the adapter 616 to the fluid container 14 and the breast pump assembly 12, the fluid chamber 52 defined by the bag 50 of the fluid container 14, can be in at least intermittent fluid communication with the outlet passage 28 defined by the outlet structure 26 of the breast pump assembly 12.

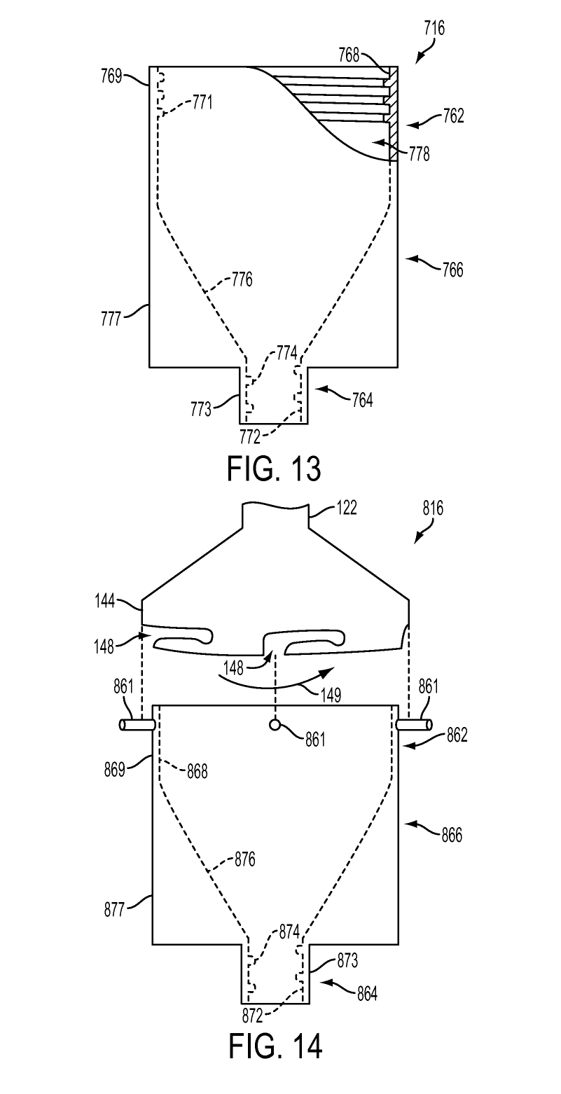

FIG. 13 illustrates an adapter 716 according to another embodiment. The adapter 716 can include a first connector portion 762, a second connector portion 764, and a transition portion 766, which can extend between the first connector portion 762 and the second connector portion 764. The first connector portion 762 can include an inner surface 768 and an outer surface 769. Unlike the first connector portion 62 of adapter 16, which is shown to include a plurality of external threads 70, the first connector portion 762 of adapter 716 can include a plurality of internal threads 771. The second connector portion 764 can include an inner surface 772, an outer surface 773, and a plurality of internal threads 774. The transition portion 766 can include an inner surface 776 and an outer surface 777. The inner surface 776 of the transition portion 766 can taper inwardly toward the inner surface 772 of the second connector portion 764. However, unlike the inner surface 76 of the transition portion 66 of adapter 16, the inner surface 776 can have a frusto-conical shape. As shown in FIG. 13, the adapter 716 does not have a uniform wall thickness. In particular, the wall thickness of the transition portion 766 of connector 716 is shown to vary. At least the first connector portion 762 and the transition portion 766 can cooperate to define an interior chamber 778, which can be configured to receive fluid, for example, breast milk.

The plurality of internal threads 774 of the second connector portion 764 can be engaged with the plurality of external threads 58 of the spout 56 of fluid container 14, to connect the adapter 716 to the fluid container 14, such that the interior chamber 778 defined by the adapter 716 is in fluid communication with the fluid chamber 52 defined by the bag 50 of the fluid container 14. The internal threads 771 of the first connector portion 762 can engage mating external threads of a component of a breast pump assembly to connect the adapter 716 to the breast pump assembly. For example, in one embodiment, the collar 44 of the breast pump assembly 12 can include a plurality of external threads (not shown) instead of the plurality of internal threads 46, which can engage the plurality of internal threads 771 of the first connector portion 762, such that the interior chamber 778 defined by the adapter 716 and the fluid chamber 52 defined by the fluid container 14 are in at least intermittent fluid communication with the outlet passage 28 defined by the outlet structure 26, during operation of the breast pump (e.g., 38, 42) of the breast pump assembly 12.

FIG. 14 illustrates an adapter 816 according to another embodiment, and a portion of a breast pump assembly according to another embodiment. The adapter 816 can include a first connector portion 862, a second connector portion 864 and a transition portion 866, which can extend between the first connector portion 862 and the second connector portion 864. The first connector portion 862 can include an inner surface 868 and an outer surface 869. Unlike adapter 16, the first connector portion 862 can include a plurality of circumferentially spaced pins 861, in lieu of the plurality of external threads 70 of the first connector portion 62 of the adapter 16. The plurality of circumferentially spaced pins 861 can extend outwardly from the outer surface 869 and can connect the adapter 816 to a collar 144 (FIG. 14) of the breast pump assembly, as subsequently described. The second connector portion 864 can include an inner surface 872, an outer surface 873, and a plurality of internal threads 874. The transition portion 866 can include an inner surface 876 and an outer surface 877. The inner surface 876 of the transition portion 866 can taper inwardly toward the inner surface 872 of the second connector portion 864. Unlike the inner surface 76 of the transition portion 66 of the adapter 16, the inner surface 876 of the transition portion 866 of adapter 816 can have a frusto-conical shape. At least the first connector portion 862 and the transition portion 866 can define an interior chamber (not shown), which can be configured to receive fluid, for example, breast milk.

The adapter 816 can be connected to each of the fluid container 14 and the breast pump assembly that includes collar 144. In one embodiment, the plurality of internal threads 874 of the second connector portion 864 can engage the plurality of external threads 58 of spout 56 of the fluid container 14, such that the interior chamber defined by the adapter 816 is in fluid communication with the fluid chamber 52 defined by the bag 50 of fluid container 14. The collar 144 of the breast pump assembly can have a different configuration than the collar 44 of the breast pump assembly 12. For example, the collar 144, which can be integral with a housing 122, can define a plurality of circumferentially spaced slots 148. Each of the slots 148 can be configured to receive a respective one of the pins 861 of the first connector portion 862, and to releasably lock the respective pin 861 in position, to connect the adapter 816 to collar 144.

The adapter 816 can be connected to the collar 144 by aligning each of the pins 861 with an entrance of a respective one of the slots 148, as shown in FIG. 14 with regard to one of the pins 861 and one of the slots 148, inserting each pin 861 into the entrance of the respective slot 148, and then rotating the adapter 816 relative to the collar 144, as indicated generally by arrow 149 in FIG. 14. The breast pump assembly can be otherwise configured the same as, or similar to, the breast pump assembly 12. As a result of the connection of the adapter 816 to each of the fluid container 14 and the collar 144 of the breast pump assembly, the interior chamber defined by the adapter 816 and the fluid chamber 52 defined by the bag 50 of the fluid container 14 can be in at least intermittent fluid communication with an outlet passage (not shown) of the breast pump assembly. Pins similar to, or the same as, pins 861 can be used in conjunction with adapters having other configurations. Also, it will be appreciated that an adapter can be selectively, removably connected to a breast pump assembly in a variety of other configurations, that may or may not include threads, and/or friction fit, etc.

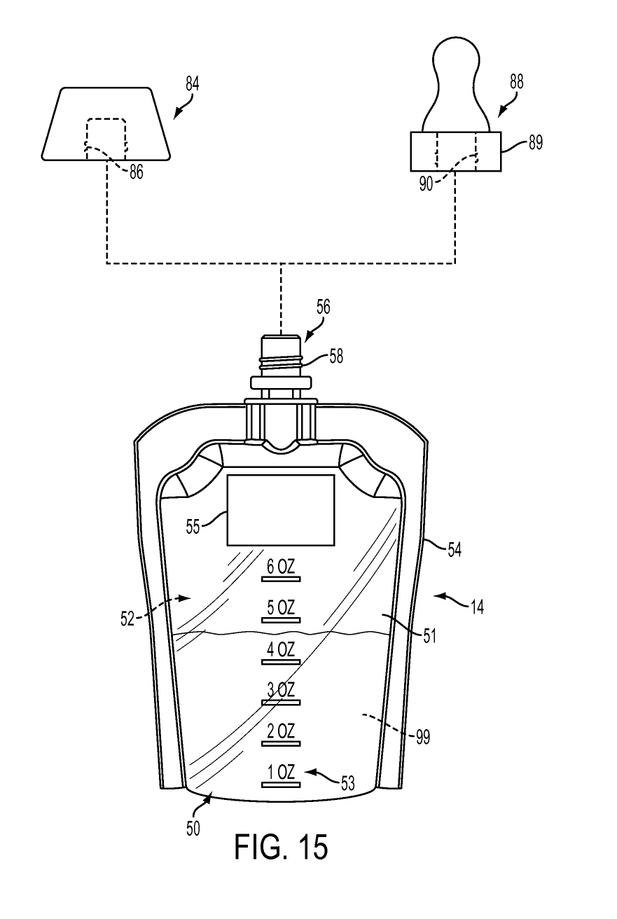

FIG. 15 illustrates the fluid container 14 of the system 10, after the fluid chamber 52 has been partially filled with breast milk 99. A first portion 51 of the bag 50 of the fluid container 14 can be transparent, to facilitate viewing the breast milk 99 within the fluid chamber 52, as shown in FIG. 15. The graduated indicia 53 of bag 50, can indicate the volume of breast milk 99 within the fluid chamber 52, as measured in ounces, for example. The bag 50 can also include a second portion 55, which can be opaque. Various information, e.g., the date the breast milk 99 was obtained, can be applied to the second portion 55 of the bag 50, such as with a pen. The sizes and shapes of the first portion 51 and the second portion 55 of bag 50 can be different than those shown. Also, the size and shape of bag 50 can be different than that shown. In other embodiments, the bag 50 can be entirely transparent, entirely translucent, entirely opaque, partially transparent and partially translucent, or partially translucent and partially opaque.

After the fluid container 14 has been filled with the desired amount of breast milk 99, the fluid chamber 52 can be closed by threading a cap, for example cap 84 shown in FIG. 15, onto the spout 56 of fluid container 14. The cap 84 can include a plurality of internal threads 86, which can engage the plurality of external threads 58 of spout 56. The fluid container 14 can then be refrigerated for later use, as subsequently described. Alternatively, in the event that it is desired to use the breast milk 99 immediately, or within a relatively short period of time after filling the fluid chamber 52 to the desired level, a nipple, for example nipple 88 shown in FIG. 15, can be threaded onto the spout 56 of the fluid container 14. In this regard, the nipple 88 can include a collar 89 that can include a plurality of internal threads 90, which can engage the plurality of external threads 58 of the spout 56.

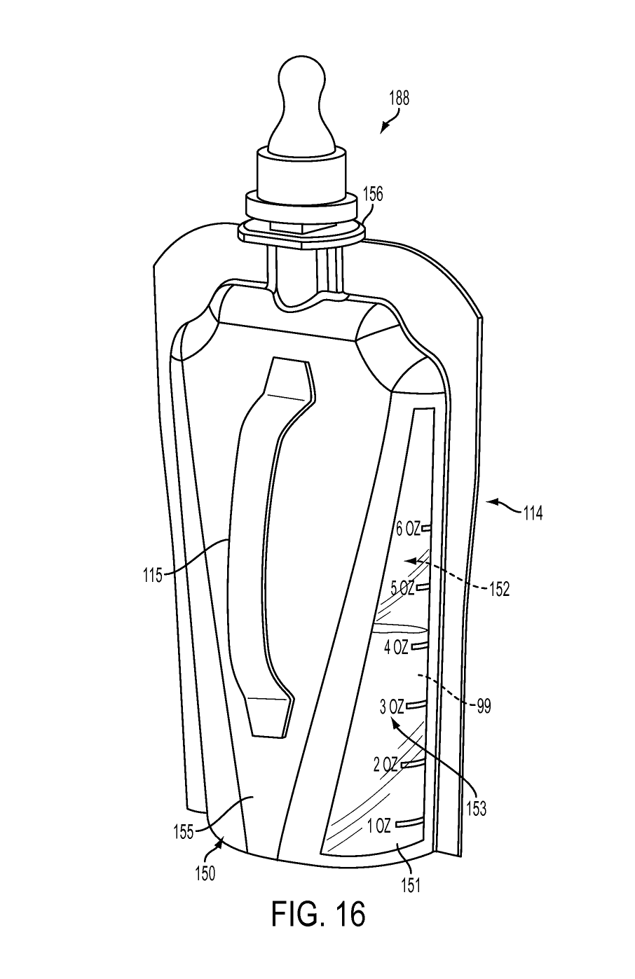

FIG. 16 illustrates a fluid container 114 according to another embodiment, which can be the same as, or similar to, the fluid container 14, except that the fluid container 114 can include a strap 115, which can be attached to a bag 150 of the fluid container 114. The strap 115 can form a loop, as shown in FIG. 16, which can facilitate holding the fluid container 114. Similar to the fluid container 14, the bag 150 of the fluid container 114 can include a first portion 151, which can be transparent, and can include a second portion 155, which can be opaque. The sizes and shapes of the first portion 151 and the second portion 155 can vary. In other embodiments, the bag 150 can be entirely transparent, entirely translucent, entirely opaque, partially transparent and partially translucent, or partially translucent and partially opaque. The fluid container 114 can also include graduated indicia 153, which can be applied to the first portion 151 to facilitate determining the volume of breast milk 99 within a fluid chamber 152 defined by the bag 150. The strap 115 can be attached to the second portion 155 of the bag 150, such that the strap 115 does not interfere with viewing the volume of breast milk 99 within the fluid chamber 152. It will be appreciated that one or more straps can be provided in any of a variety of other suitable configurations (e.g., one or more finger loops) and locations. The fluid container 114 can include a spout 156 that can be attached to the bag 150 of fluid container 114. The spout 156 can include a plurality of external threads (not shown) and can define a flow passage (not shown) that can be in fluid communication with fluid chamber 152. A nipple 188 can be threaded onto the spout 156, as shown in FIG. 16, to facilitate dispensing of the breast milk 99 from the fluid chamber 152. The strap 115 can assist a caretaker in holding the fluid container 114 during the pouring process.

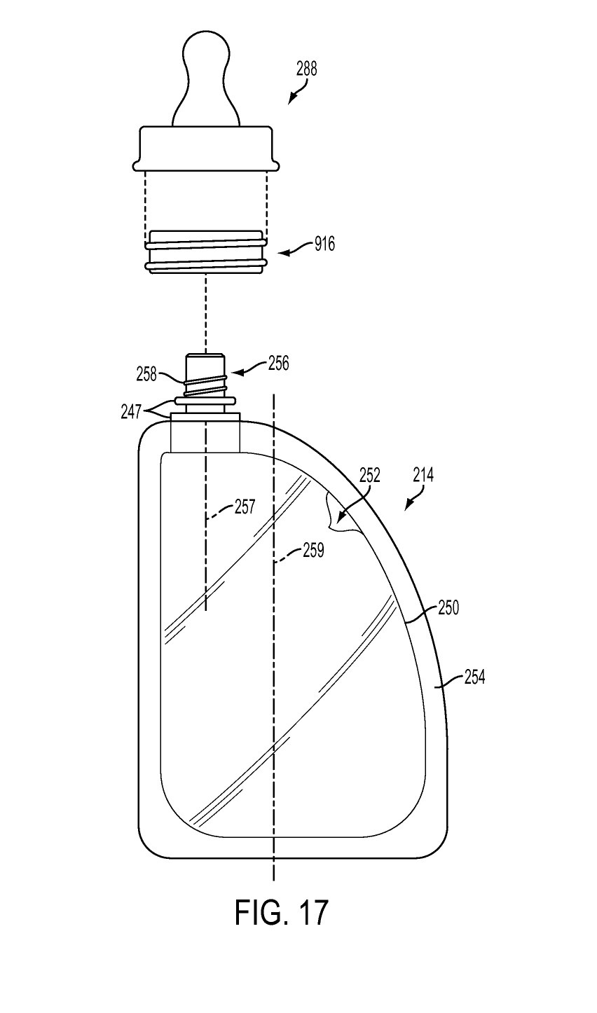

FIG. 17 illustrates a fluid container 214 according to another embodiment, in association with an adapter 916 and a nipple 288. The adapter 916 can be connected, for example threadedly connected, to each of the fluid container 214 and the nipple 288 such that breast milk (not shown) contained within the fluid container 214 can be dispensed through the nipple 288. The fluid container 214 can include a bag 250 and a spout 256 attached to the bag 250. The bag 250 can define a fluid chamber 252 (FIGS. 17 and 20) and the spout 256 can define a fluid passage 260 (FIG. 28). The bag 250 can be at least partially collapsible, and can be expandable, such that the fluid chamber 252 can have a predetermined, maximum volume. The fluid chamber 252 defined by the bag 250 of fluid container 214 can be in fluid communication with the fluid passage 260 defined by the spout 256, and can be otherwise sealed, such that a caretaker using the fluid container 214 cannot create an opening (other than through the spout 256) without destroying the bag 250.

The fluid container 214 can also include a perimeter flange 254, which can at least partially surround the bag 250. The bag 250 can include at least one plastic sheet, using any suitable plastic material. In one embodiment, the bag 250 can include two plastic sheets, which can be attached to one another and to the spout 256, e.g., using a heat-sealing process and/or adhesive. The two plastic sheets can cooperate to define the interior chamber 252 and can form at least a portion of the perimeter flange 254.

At least a portion of the bag 250 can be transparent. In one embodiment, the bag 250 can be entirely transparent, as shown in FIGS. 17, 19 and 20, which can facilitate viewing breast milk within the fluid chamber 252. In another embodiment, the fluid container 214 can include a first, transparent portion and a second, opaque portion. Various information, such as the date any breast milk within the bag 250 of fluid container 214 was obtained, can be applied to the opaque portion, such as with a pen. The bag 250 can also include graduated indicia (not shown), which can be located on a transparent portion of bag 250, and can indicate the volume of fluid, e.g., breast milk, within the fluid chamber 252, as measured in ounces or milliliters, for example. In other embodiments, the bag 250 can be entirely translucent, entirely opaque, partially transparent and partially translucent, or partially translucent and partially opaque.

The bag 250 can include a longitudinal centerline 259, as shown in FIG. 17. The spout 256 can include a plurality of external threads 258 and at least one flange 247. In one embodiment, the spout 256 can include two of the flanges 247, which can be longitudinally spaced, as shown in FIG. 17. The size and shape of the flanges 247, as well as the longitudinal spacing between the flanges 247, can be different than that shown. The spout 256 can be offset from the longitudinal centerline 259 of the bag 250. The spout 256 can include a longitudinal centerline 257, which can be laterally spaced from the longitudinal centerline 259 of the bag 250. In one embodiment, the longitudinal centerline 257 of the spout 256 can be parallel with the longitudinal centerline 259 of the bag 250, as shown in FIG. 17. This configuration can provide enhanced ergonomics when feeding an infant from the fluid container 214 and can at least reduce residual milk within the fluid container 214 after feeding, for example, as compared to at least some fluid containers that do not include offset spouts.

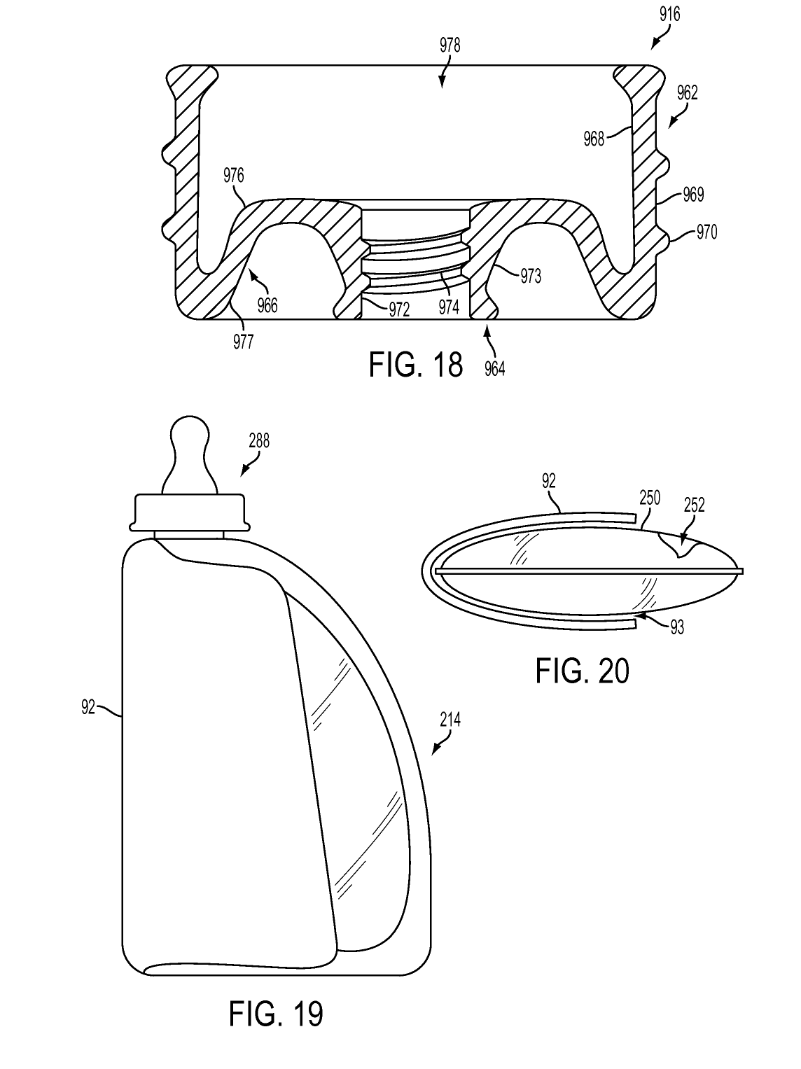

Referring to FIG. 18, the adapter 916 can include a first connector portion 962, a second connector portion 964, and a transition portion 966 that can extend between the first connector portion 962 and the second connector portion 964. The first connector portion 962 can include an inner surface 968, an outer surface 969, and a plurality of external threads 970, which can extend away from the outer surface 969. The second connector portion 964 can include an inner surface 972, an outer surface 973, and a plurality of internal threads 974. The transition portion 966 can include an inner surface 976 and an outer surface 977. At least the first connector portion 962 and the transition portion 966 can define an interior chamber 978.

As shown in FIG. 18, a pitch diameter of the external threads 970 of the first connector portion 962 can be larger than a pitch diameter of the internal threads 974 of the second connector portion 964. In one embodiment, as shown in FIG. 18, the pitch diameter of the external threads 970 can be significantly larger than the pitch diameter of the internal threads 974. The relative pitch diameters of the external threads 970 and internal threads 974 can permit the interconnection of the nipple 288 and spout 256, via the adapter 916 even though the external threads 258 of spout 256 can be significantly smaller than the internal threads (not shown) of the nipple 288. This permits fluid to be efficiently dispensed from the fluid chamber 252 defined by the bag 250 of fluid container 214, through the nipple 288. Adapter 916 allows use of the fluid container 214 with a nipple (e.g., 288) having the same thread configuration as the internal threads 46 of the collar 44 of breast pump assembly 12. Alternatively, other adapters (e.g., 16) can be used in lieu of adapter 916.

FIGS. 19 and 20 illustrate a receptacle 92 according to one embodiment, which can be used in association with the fluid container 214, to facilitate holding and controlling the fluid container 214 during feeding of an infant. The receptacle 92 can be formed from a rigid, or semi-rigid material, e.g., any suitable plastic material, using any suitable molding process. As shown in FIG. 20, the receptacle 92 can define an interior cavity 93, which can be configured to receive a portion of the bag 250 of the fluid container 214, e.g., a shape of the interior cavity 93 can be complementary with a shape of at least a portion of the bag 250. In other embodiments, receptacles can be provided that are configured other than as shown, for receiving a fluid container (e.g., 14, 114 and 214), and can be releasably connected to a fluid container (e.g., 14, 114 and 214). Use of a receptacle such as receptacle 92 can enable a caretaker to hold the associated fluid container (e.g., 214) without squeezing breast milk out of the fluid container (e.g., 214), which may otherwise occur in view of the flexible construction of the bag of the fluid container (e.g., bag 250 of fluid container 214).

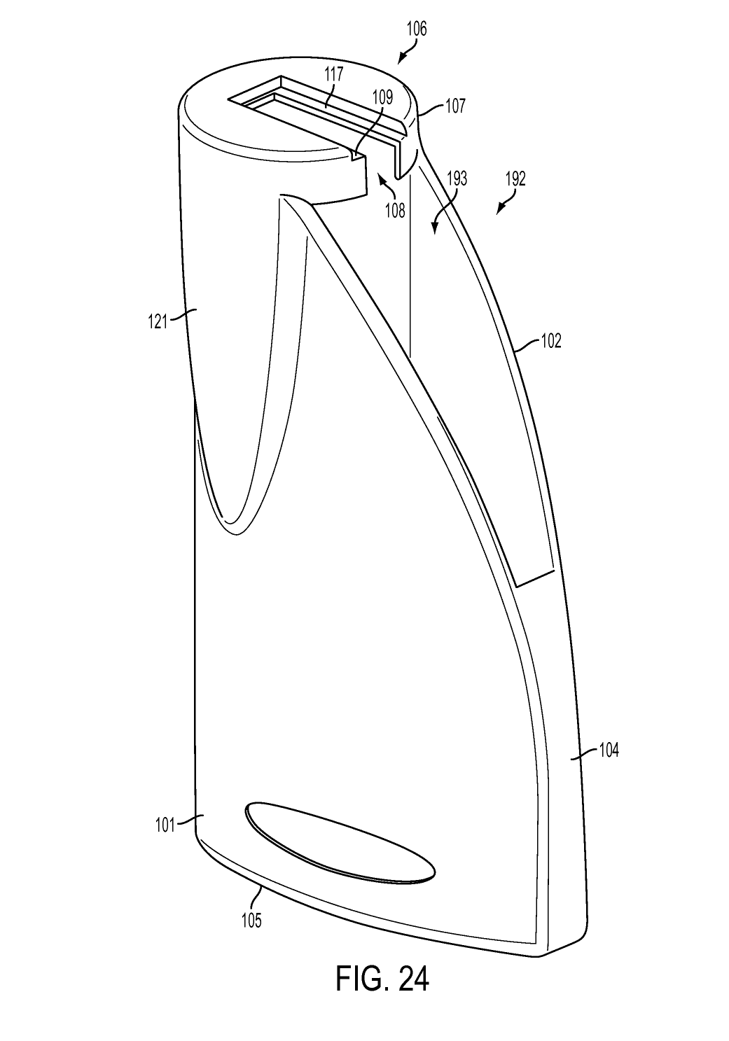

FIGS. 24-26 illustrate a receptacle 192 according to another embodiment. The receptacle 192 can define an interior cavity 193 that can be configured to receive a fluid container, e.g., 214, such that at least a portion of a bag of a fluid container, e.g., bag 250 of fluid container 214, can be positioned within the interior cavity 193. The receptacle 192 can be formed from a rigid, or semi-rigid material, e.g., any suitable plastic material, using any suitable molding process. The configuration and rigidity of receptacle 192 can facilitate holding an associated fluid container (e.g., 214) during feeding of an infant, without squeezing breast milk out of the associated fluid container (e.g., 214).

The receptacle 192 can include a first side wall 101, a second side wall 102 spaced from the first side wall 101, and a first end wall 103, which can be integral with, and can extend between, the first side wall 101 and the second side wall 102. The receptacle 192 can also include a second end wall 104, which can be integral with, and can extend between, the first side wall 101 and the second side wall 102. In one embodiment, the receptacle 192 can include a first end 105 and a fluid-container-support structure 106, which can be longitudinally spaced from the first end 105. The fluid-container-support structure 106 can be integral with at least the first side wall 101 and the second side wall 102. In one embodiment, the fluid-container-support structure 106 can be integral with each of the first side wall 101, the second side wall 102, and the first end wall 103. In one embodiment, the first side wall 101, the second side wall 102, the first end wall 103, the second end wall 104 and the fluid-container-support structure 106 can be integrally formed, for example, from any suitable plastic material, as a unitary structure.

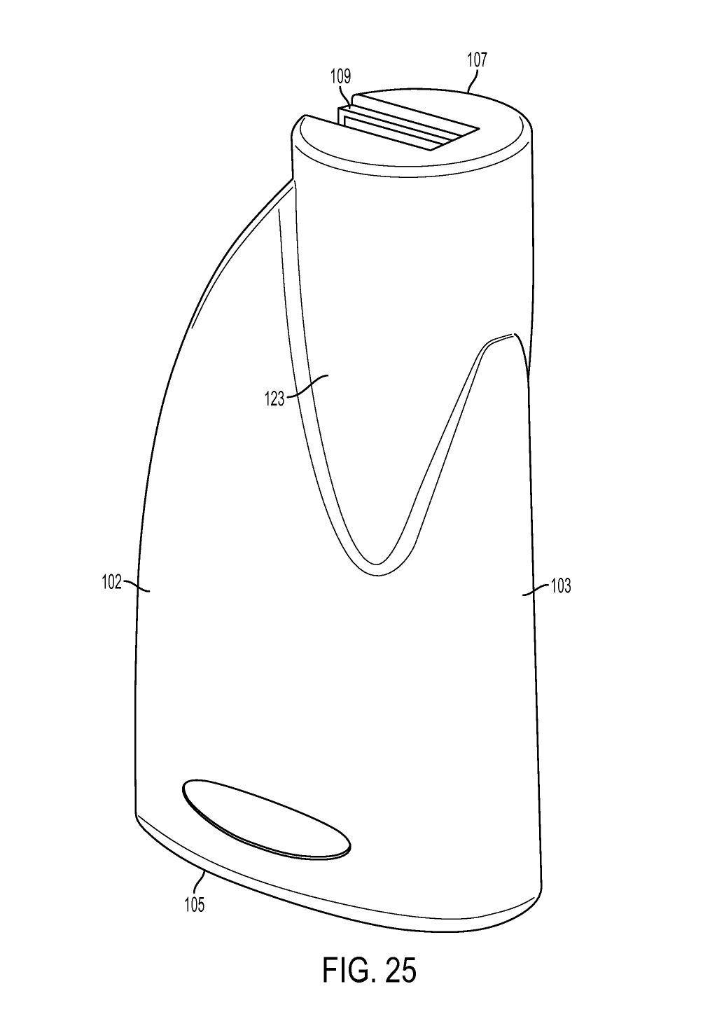

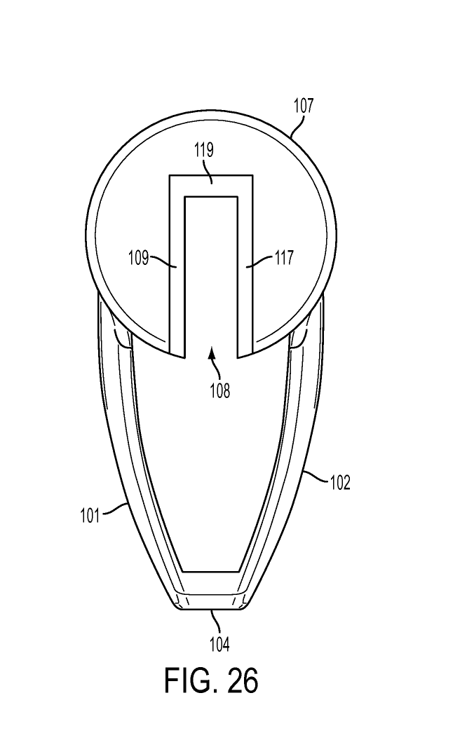

The fluid-container-support structure 106 can include an end portion 107. The end portion 107 can define an aperture 108 (FIGS. 24 and 26), which can communicate with the interior cavity 193. The end portion 107 can include a first rail 109 and a second rail 117, which can be spaced from the first rail 109. As shown in FIG. 26, each of the first rail 109 and the second rail 117 can be adjacent to the aperture 108. The end portion 107 can also include a ledge 119 (FIG. 26), which can extend between the first rail 109 and the second rail 117. The fluid-container-support structure 106 can also include a first side portion 121 and a second side portion 123. Each of the first side portion 121 and the second side portion 123 can be integral with, and can extend away from, the end portion 107 of the fluid-container-support structure 106. The first side portion 121 can be integral with, and can protrude outwardly from, the first side wall 101 of the receptacle 192, as shown in FIG. 24. The second side portion 123 of the fluid-container-support structure 106 can be integral with, and can protrude outwardly from, the second side wall 102 of the receptacle 192, as shown in FIG. 25.

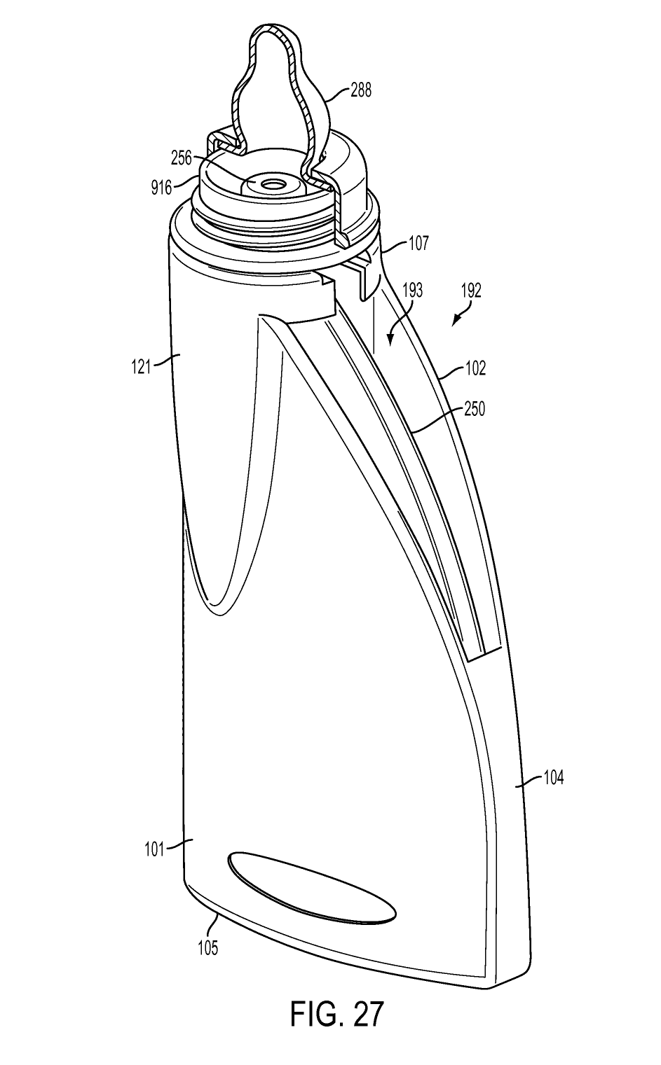

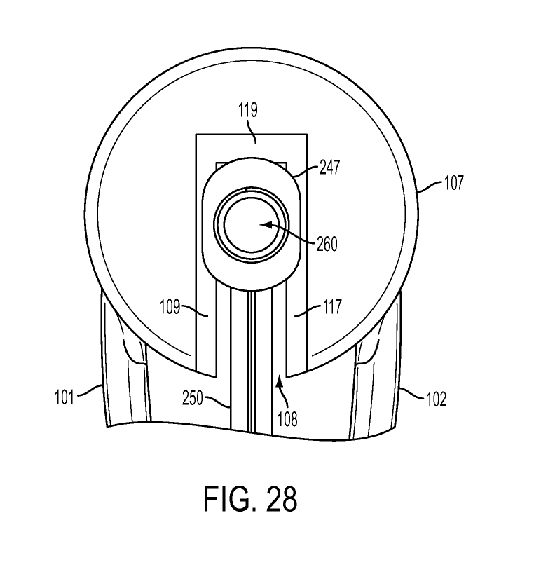

FIG. 27 illustrates the receptacle 192 in association with the fluid container 214, adapter 916, and nipple 288, which are also shown in FIGS. 17-20. As shown in FIG. 27, the second end wall 104 of the receptacle 192 can extend from the first end 105 of receptacle 192 toward the end portion 107 of the fluid-container-support structure 106, but can be spaced from the end portion 107 to provide access to the interior cavity 193 defined by the receptacle 192. This access can permit a caretaker to insert a portion of the fluid container 214 into the interior cavity 193. For example, a caretaker can insert a portion of the fluid container 214 that is spaced from the spout 256 into the interior cavity 193, and can then rotate the fluid container 214 such that one of the flanges 247 of the spout 256 engages each of the first rail 109 and the second rail 117 of the end portion 107 of the fluid-container-support structure 106 of receptacle 192, as shown in FIG. 28. The flange 247 can slidingly engage each of the first rail 109 and the second rail 117, such that the fluid container 214 can be moved toward the ledge 119 of the end portion 107 of the fluid-container-support structure 106. The flange 247 can contact the ledge 119, or alternatively, can be spaced from the ledge 119.

The first end 105, which can include an end surface of each of the first side wall 101, the second side wall 102, the first end wall 103, and the second end wall 104, can be configured to rest upon a support surface, for example a countertop or a table. When the receptacle 192 is positioned in an upright orientation, as shown in FIGS. 24, 25, and 27, the fluid container 214 can be suspended from the first rail 109 and the second rail 117. The adapter 916 can then be threaded onto the spout 256 of the fluid container 214, and the nipple 288 can be threaded onto the adapter 916. In this assembled configuration, fluid, e.g., breast milk, can be dispensed from the bag 250 of the fluid container 214, through the passage 260 defined by the spout 256, and through the nipple 288. The receptacle 192 can have an ergonomic configuration that can assist a caretaker when feeding an infant. For example, the fingers of a caretaker's hand can wrap around the first end wall 103 of the receptacle 192 and can engage one of the first side wall 101 and the second side wall 102. The thumb of the caretaker's hand can engage the other one of the first side wall 101 and the second side wall 102, and can be positioned adjacent a respective one of the first side portion 121 and the second side portion 123 of the fluid-container-support structure 106, which can protrude outwardly from the first side wall 101 and the second side wall 102, respectively, such as to enhance ergonomics and/or grip. This grip of the receptacle 192 can enhance a caretaker's control of the receptacle 192.

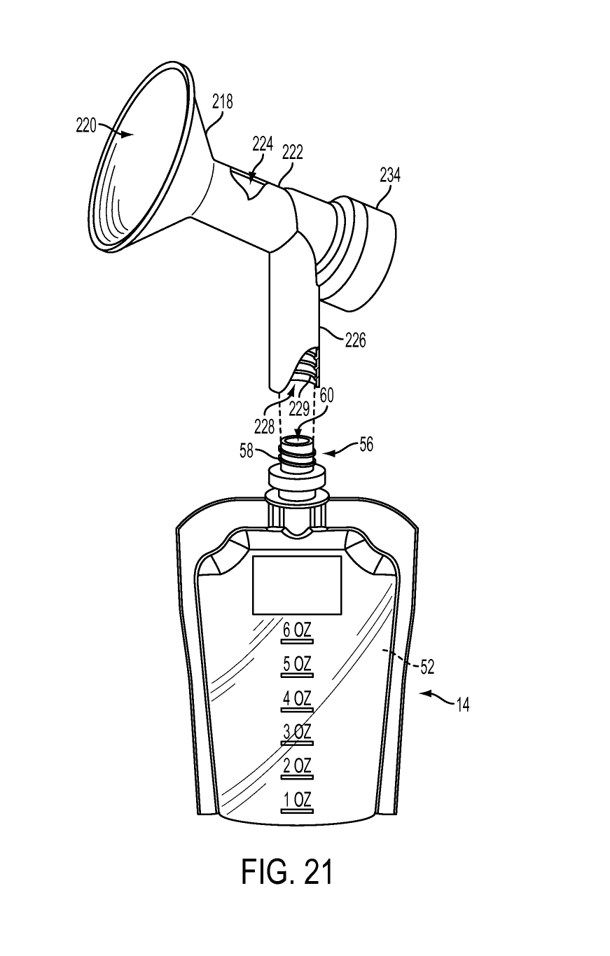

FIG. 21 illustrates the fluid container 14 in association with a portion of a breast pump assembly according to another embodiment. The breast pump assembly can include a hood 218, which can define an extraction chamber 220, and a first housing 222. The first housing 222 can be integral with the hood 218 and can define a flow passage 224, which can be in fluid communication with the extraction chamber 220. The breast pump assembly can also include a second housing 234 that can be integral with the first housing 222 and can define a vacuum chamber (not shown), which can be in fluid communication with the flow passage 224, and a breast pump (not shown). The breast pump assembly can also include an outlet structure 226, which can be integral with at least one of the first housing 222 and the second housing 234 and can include a plurality of internal threads 229. The outlet structure 226 can define an outlet passage 228, which can be in fluid communication with the extraction chamber 220 and the vacuum chamber via the flow passage 224. The outlet structure 226 can also include a valve member (not shown) which can intermittently open and close, in response to operation of the breast pump of the breast pump assembly, to intermittently permit expressed breast milk to flow through, and discharge from, the outlet passage 228. The internal threads 229 of the outlet structure 226 can engage the external threads 58 of the spout 56 of fluid container 14, to connect the fluid container 14 to the outlet structure 226, such that the fluid chamber 52 defined by the fluid container 14 is in at least intermittent fluid communication with the outlet passage 228 during operation of the breast pump. Accordingly, breast milk can be pumped out of the outlet passage 228 defined by the outlet structure 226, through the fluid passage 60 defined by the spout 56 of the fluid container 14 and into the fluid chamber 52 defined by the bag 50 of the fluid container 14, without the use of an adapter or other intervening structure.

A method of expressing breast milk according to one embodiment, can be illustrated with respect to the breast pump assembly 12, fluid container 14 and adapter 16, and with reference to FIGS. 1, 2, 15, 22 and 23. The adapter 16 can be connected to each of the fluid container 14 and the breast pump assembly 12, for example by threadedly engaging the internal threads 74 of the second connector portion 64 of adapter 16 with the external threads 58 of the spout 56 of fluid container 14, and threadedly engaging the external threads 70 of the first connector portion 62 of adapter 16 with the internal threads 46 of the collar 44 of breast pump assembly 12. The connection of the adapter 16 to the fluid container 14 can establish fluid communication between the interior chamber 78 defined by the adapter 16 and the fluid chamber 52 defined by the fluid container 14.

The connection of the adapter 16 with the collar 44 of the breast pump assembly 12 can provide at least intermittent fluid communication between the interior chamber 78 defined by the adapter 16 and the outlet passage 28 defined by the outlet structure 26 of the breast pump assembly, i.e., the interior chamber 78 can be in fluid communication with the outlet passage 28 when the valve member 32 of the outlet structure 26 is open. The valve member 32 can be intermittently opened and closed during operation of the breast pump (e.g., 38, 42) of the breast pump assembly 12. Accordingly, due to the connection of the adapter 16 to each of the fluid container 14 and the breast pump assembly 12, the fluid chamber 52 defined by the fluid container 14 can be in at least intermittent fluid communication with the outlet passage 28 defined by the outlet structure 26 of the breast pump assembly 12, during operation of the breast pump (e.g., 38, 42) of the breast pump assembly 12. As a result, expressed breast milk 99 can intermittently flow from the breast pump assembly 12 through adapter 16 and into the fluid chamber 52 defined by the fluid container 14.

This process can be continued until a desired volume of breast milk 99 has been pumped into the fluid chamber 52. The transparent nature of the first portion 51 of the bag 50 of fluid container 14 can facilitate determining when the desired volume of breast milk 99 has been obtained. If a caretaker desires to feed an infant either immediately after obtaining the breast milk 99, or within a relatively short period of time, a nipple, e.g., the nipple 88 shown in FIG. 15 or the nipple 288 plus the adapter 916 shown in FIG. 17, can be connected to the spout 56 of the fluid container 14 such that the breast milk 99 can be dispensed through the nipple, for example nipple 88 or nipple 288, to the infant.

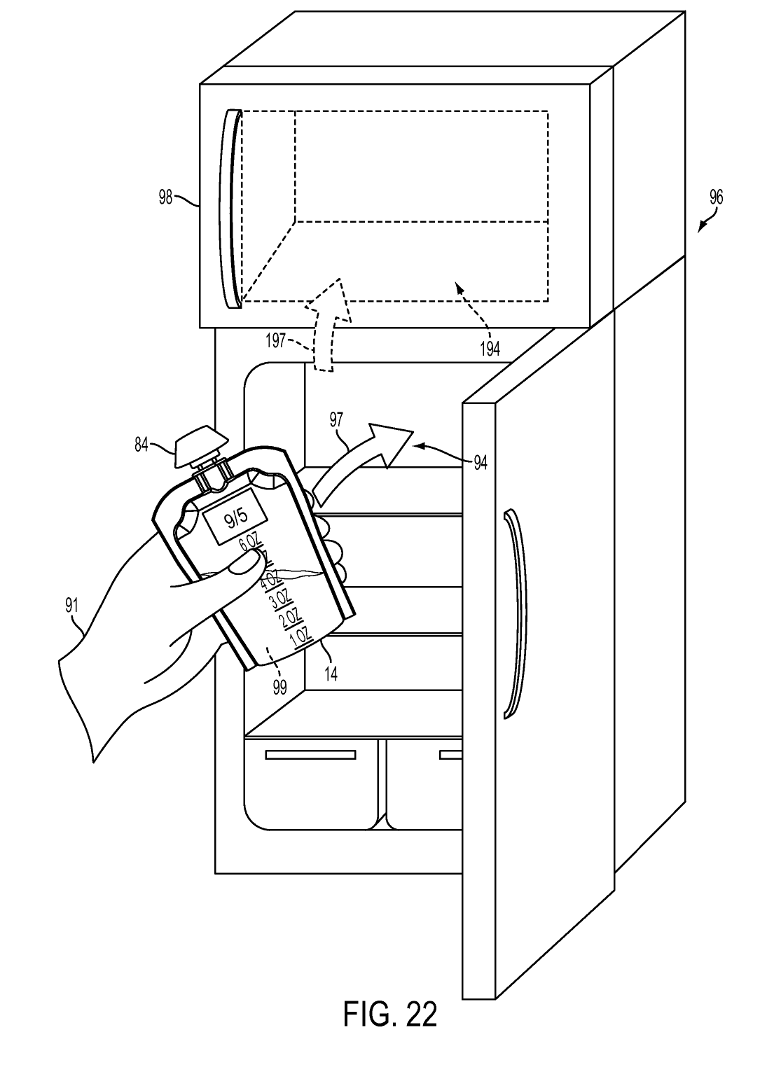

Alternatively, if it is desired to store the breast milk 99 for a relatively longer period of time, a cap, e.g., the cap 84, can be threaded onto the spout 56 of the fluid container 14, to close the fluid chamber 52. A caretaker can then place the fluid container 14 within a refrigerated space, for example, a refrigerated space 94 of a refrigerator 96 (FIG. 22). FIG. 22 illustrates a hand 91 of a caretaker grasping the fluid container 14, which is shown to be partially filled with breast milk 99, and placing the fluid container 14 into the refrigerated space 94 of refrigerator 96, as indicated generally by arrow 97 in FIG. 22. Alternatively, for longer term storage, the fluid container 14 can be placed within a refrigerated space 194 of a freezer 98 of the refrigerator 96, as indicated generally by arrow 197, shown in dashed lines in FIG. 22.

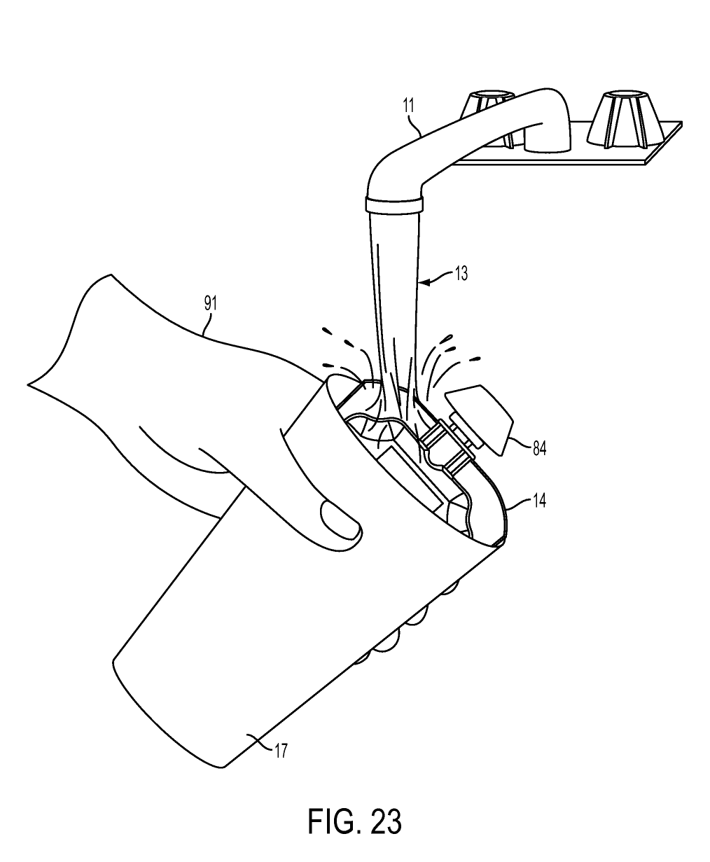

Subsequently, when a caretaker desires to dispense the breast milk 99 from the fluid container 14, e.g., to feed an infant, the caretaker can heat the fluid container 14 to increase the temperature of the breast milk 99 within the fluid container 14. For example, as illustrated in FIG. 23, a caretaker can place the fluid container 14 at least partially within a receptacle 17, which can be a cup or any other suitable receptacle, and can hold the receptacle 17 and fluid container 14 under a water faucet 11, such that a stream of water 13 discharging from the water faucet 11 can flow onto and over the fluid container 14, to warm the temperature of the breast milk 99 within the fluid container 14.

The use of an adapter (e.g., 16, 116, 216, 316, 416, 516, 616, and 916) to interconnect a fluid container (e.g., 14, 114 and 214) and a breast pump assembly (e.g., 12) can result in various advantages. Adapters having other configurations (e.g., 716 and 816) can also be advantageously used with a fluid container (e.g., 14, 114 and 214) and a breast pump assembly that can be configured, at least in part, differently than the breast pump assembly 12. These advantages can include increasing the quantity of useable, expressed breast milk, which is recognized to be a very precious and valuable commodity. For example, typical processes for expressing and storing breast milk for subsequent consumption, include pumping breast milk into a bottle, pouring the breast milk out of the bottle into a flexible bag, which does not include a spout, storing the bag in a refrigerated space, removing the bag and heating the bag to raise the temperature of the breast milk within the bag, and pouring the breast milk out of the bag, which can have a relatively wide mouth or opening, into a bottle for use. Pouring the breast milk from the initial bottle into the bag can result in residual breast milk remaining in the bottle, e.g., breast milk that adheres to the side wall and/or the bottom of the bottle. This residual breast milk is not usable. Residual breast milk may also remain within the flexible bag when the breast milk within the bag is poured into a bottle for use. Furthermore, it will be appreciated that spillage may occur when the breast milk is poured from the bag into the bottle, due to the size and shape of the opening in the bag, the lack of a spout attached to the bag, and the difficulty in controlling the bag during the pouring process since the bag remains flexible, even when partially filled with breast milk.

In contrast, pumping breast milk into a fluid container that is suitable for storage, for example fluid containers 14, 114 and 214, eliminates the step of pouring expressed breast milk into a storage bag, after pumping the breast milk into a bottle, and therefore increases the quantity of useable breast milk by eliminating any residual, or non-useable, breast milk within the bottle. This also eliminates the need for cleaning and sterilizing the bottle. Additionally, use of a fluid container with a spout, for example fluid container 14 that includes a threaded spout 56, can enhance the ability to pour the breast milk out of the fluid container into a bottle while reducing, or eliminating, spilled breast milk, and can enhance the ability to control the quantity of breast milk poured into the bottle. The use of a fluid container with an offset spout, for example fluid container 214 with offset spout 256, can further enhance the ability to pour the breast milk out of the fluid container (e.g., 214), or to feed an infant with a nipple attached to the fluid container, while avoiding spillage or leaving residual breast milk in the fluid container.

The use of a receptacle made of a rigid or semi-rigid material in conjunction with a fluid container, for example receptacles 92, 192 which can receive and at least partially surround the bag of a fluid container, for example bag 250 of fluid container 214, can enhance the ability to hold and control the fluid container and can therefore enhance the ability to pour breast milk out of the fluid container while avoiding spillage. Use of adapters according to various embodiments, for example (but not limited to) adapters 16 and 916, can permit the use of a fluid container having a flexible bag which can be at least partially collapsible, and expandable, with certain components (e.g., a hood defining an extraction chamber, a breast pump, threaded collar, and nipple) of an otherwise conventional system for expressing breast milk.

While various embodiments of an adapter, a fluid container, a system for expressing breast milk that includes an adapter, subassemblies of such a system, and a method of expressing breast milk, have been illustrated by the foregoing description and have been described in considerable detail, it is not intended to restrict or in any way limit the scope of the appended claims to such detail. Additional modifications will be readily apparent to those skilled in the art.

1. An adapter comprising:
a first connector portion comprising a first inner surface and a first outer surface;
a second connector portion comprising a second inner surface, a second outer surface, and a plurality of internal threads; and
a transition portion comprising a third inner surface and a third outer surface, the transition portion extending between the first connector portion and the second connector portion, the third inner surface of the transition portion tapering inwardly toward the second inner surface of the second connector portion; wherein
at least the first connector portion and the transition portion cooperate to define an interior chamber configured to receive fluid;
the first connector portion is configured for connection to a pump assembly to facilitate establishing at least intermittent fluid communication between an outlet passage of a pump assembly and the interior chamber; and
the second connector portion is configured for connection, via the plurality of internal threads, to a spout of a fluid container to facilitate establishing fluid communication between the interior chamber and a chamber defined by a fluid container.

2. The adapter of claim 1, wherein:
the first connector portion further comprises a plurality of external threads.

3. The adapter of claim 2, wherein:
the third inner surface of the transition portion is generally cup-shaped.

4. The adapter of claim 2, wherein:
the third inner surface of the transition portion blends smoothly with at least the first inner surface of the first connector portion.

5. The adapter of claim 2, further comprising:
a plurality of circumferentially spaced fins.

6. The adapter of claim 5, wherein:
each of the fins extends outwardly from at least one of the transition portion and the second connector portion.

7. The adapter of claim 6, wherein:
each of the fins comprises a linear, distal surface extending outwardly from the annular distal surface of the second connector portion, such that the linear, distal surface is coplanar with the annular distal surface.

8. The adapter of claim 6, wherein:
each of the fins extends distally beyond the annular distal surface of the second connector portion.

9. The adapter of claim 8, wherein:
each of the fins comprises an arcuate, distal surface extending outwardly from the second connector portion.

10. The adapter of claim 2, wherein:
the plurality of external threads comprises a first pitch diameter;
the plurality of internal threads comprises a second pitch diameter; and
the first pitch diameter is greater than the second pitch diameter.

11. The adapter of claim 10, further comprising:
a longitudinal centerline; wherein
the first connector portion, the second connector portion, and the transition portion are integrally formed as a unitary structure; and
the longitudinal centerline centrally bisects the first connector portion, the second connector portion and the transition portion.

12. The adapter of claim 11, wherein:
the first connector portion, the second connector portion, and the transition portion are integrally formed from a polymeric material;
the first connector portion further comprises an annular proximal surface; and
the second connector portion further comprises an annular distal surface.

13. The adapter of claim 6, wherein:
the circumferentially spaced fins, the first connector portion, the second connector portion, and the transition portion, are integrally formed from a polymeric material as a unitary structure.

14. (canceled)

15. (canceled)

16. (canceled)

17. (canceled)

18. (canceled)

19. (canceled)

20. (canceled)

21. A subassembly of a system for expressing breast milk, the subassembly comprising:
an adapter comprising a first connector portion, a second connector portion, and a transition portion extending between the first connector portion and the second connector portion, the first connector portion comprising a first inner surface and a first outer surface, the second connector portion comprising a second inner surface, a second outer surface, and a plurality of internal threads, the transition portion comprising a third inner surface and a third outer surface, the third inner surface of the transition portion tapering inwardly toward the second inner surface of the second connector portion, at least the first connector portion and the transition portion cooperating to define an interior chamber; and
a fluid container comprising a bag and a spout attached to the bag, the bag being at least partially collapsible, and expandable, the bag defining a fluid chamber, the spout comprising a plurality of external threads and defining a flow passage in fluid communication with the fluid chamber; wherein
the first connector portion of the adapter is configured for connection to a pump assembly to facilitate establishing at least intermittent fluid communication between an outlet passage of a pump assembly and the interior chamber of the adapter; and
the plurality of internal threads of the second connector portion of the adapter is configured for engagement with the plurality of external threads of the spout of the fluid container to facilitate establishing fluid communication between the interior chamber defined by the adapter and the fluid chamber defined by the bag of the fluid container, via the flow passage defined by the spout of the fluid container.

22. The subassembly of claim 21, wherein:
the first connector portion of the adapter further comprises a plurality of external threads;
the third inner surface of the transition portion of the adapter is generally cup-shaped; and
the third inner surface of the transition portion of the adapter blends smoothly with at least the first inner surface of the first connector portion of the adapter.

23. The subassembly of claim 22, wherein:
the adapter further comprises a plurality of circumferentially spaced fins;
the first connector portion of the adapter further comprises an annular proximal surface; and
the second connector portion of the adapter further comprises an annular distal surface.

24. The subassembly of claim 23, wherein:
each of the fins of the adapter extends outwardly from at least one of the transition portion of the adapter and the second connector portion of the adapter.

25. The subassembly of claim 24, wherein:
each of the fins of the adapter comprises a linear, distal surface extending outwardly from the annular distal surface of the second connector portion of the adapter such that the linear, distal surface is coplanar with the annular distal surface.

26. The subassembly of claim 24, wherein:
each of the fins of the adapter extends distally beyond the annular distal surface of the second connector portion of the adapter.

27. The subassembly of claim 23, wherein:
the plurality of external threads of the first connector portion of the adapter comprises a first pitch diameter;
the plurality of internal threads of the second connector portion of the adapter comprises a second pitch diameter; and
the first pitch diameter is greater than the second pitch diameter.

28. The subassembly of claim 27, wherein:
the first connector portion of the adapter, the second connector portion of the adapter, and the transition portion of the adapter, are integrally formed from a polymeric material as a unitary structure.

29. The subassembly of claim 22, further comprising:
a hood defining an extraction chamber;
a housing integral with the hood, the housing defining at least one flow passage;
a collar integral with the housing, the collar comprising a plurality of internal threads configured for engagement with the plurality of external threads of the first connector portion of the adapter; and
an outlet structure integral with the housing and defining an outlet passage, the at least one flow passage defined by the housing establishing fluid communication between the outlet passage and the extraction chamber.

30. The subassembly of claim 29, further comprising:
a second housing integral with the first housing, the second housing defining a vacuum chamber, the vacuum chamber being in fluid communication with each of the extraction chamber and the outlet passage via the at least one flow passage defined by the housing.

31. The subassembly of claim 22, further comprising:
a cap configured for engagement with the plurality of external threads of the spout of the fluid container.

32. The subassembly of claim 22, further comprising:
a nipple configured for engagement with the plurality of external threads of the spout of the fluid container.

33. (canceled)

34. (canceled)

35. (canceled)

36. (canceled)

37. (canceled)

38. (canceled)

39. (canceled)

40. (canceled)

41. (canceled)

42. (canceled)

43. (canceled)

44. (canceled)

45. (canceled)

46. (canceled)

47. (canceled)

48. (canceled)

49. (canceled)

50. (canceled)

51. (canceled)

52. (canceled)

53. (canceled)

54. (canceled)

55. A method of expressing breast milk comprising:
pumping breast milk into and through a flow passage defined by a threaded spout of a fluid container, and into a fluid chamber defined by a bag of the fluid container, the threaded spout being attached to the bag;
closing the fluid chamber by threading a cap onto the threaded spout of the fluid container; and
storing the fluid container in a refrigerated space, for later use.

56. The method of claim 55, further comprising:
removing the fluid container from the refrigerated space; and
heating the fluid container to increase the temperature of the breast milk within the fluid chamber.

57. The method of claim 56, further comprising:
replacing the cap with a nipple threaded onto the spout of the fluid container.

58. The method of claim 57, further comprising:
surrounding at least a portion of the bag of the fluid container with a receptacle to facilitate holding the fluid container, the receptacle being at least semi-rigid.

59. The method of claim 58, wherein:
pumping comprises connecting the adapter to each of a breast pump assembly and the fluid container to establish at least intermittent fluid communication between the fluid chamber defined by a bag of the fluid container and an outlet passage defined by an outlet structure of the breast pump assembly; and
the method further comprises disconnecting the adapter from the fluid container prior to closing the fluid chamber.
